(19) United States
(12) Patent Application Publication
(10) Pub. No.: US 2004/0253617 A1
(43) Pub. Date: Dec. 16, 2004

(54) Molecular typing of group B streptococci

(76) Inventors: Kong Fanrong, Westmead (AU); Gwendolyn Gilbert, Riverview (AU)

Correspondence Address: Thomas J. Kowalski, Esq., c/o FROMMER LAWRENCE & HAUG, LLP, 745 Fifth Avenue, New York, NY, 10151, US

(21) Appl. No.: 10/804,408

(22) Filed: Mar. 19, 2004

Related U.S. Application Data
(63) Continuation-in-part of application No. PCT/AU02/01281, filed on Sep. 18, 2002.

(30) Foreign Application Priority Data
Sep. 19, 2001 (AU) PR 7749

(51) Int. Cl. C12Q 1/68

(52) U.S. Cl. 435/6

(57) ABSTRACT

Molecular methods are provided for typing group B streptococci, as well as polynucleotides useful in such methods.

### FIELD OF THE INVENTION

The present invention relates to molecular methods of typing group B streptococci, as well as polynucleotides useful in such methods.

### BACKGROUND TO THE INVENTION

Group B streptococcus (GBS)—Streptococcus agalactiae—is the commonest cause of neonatal and obstetric sepsis and an increasingly important cause of septicaemia in the elderly and immunocompromised patients. The incidence of neonatal GBS sepsis has been reduced in recent years by the use of intrapartum antibiotic prophylaxis, but there are many problems with this approach. In future, vaccination is likely to be preferred and there has been considerable progress in development of conjugate polysaccharide GBS vaccines.

Before the introduction of conjugate vaccines, extensive epidemiological and other related studies will be required to assess, not only the burden of disease, but also the distribution of GBS types (including capsular polysaccharide gene serotypes, serosubtypes; protein antigen gene subtypes; mobile genetic element subtypes) to determine the optimal formulation of vaccine antigens. Type distribution based on one geographic location or small numbers of patients may not be generally applicable. Continued monitoring will be necessary to assess the suitability of combinations of GBS vaccine antigens for different target populations in different geographic locations.

Nine capsular polysaccharide GBS serotypes have been described (Harrison et al., 1998; Hickman et al., 1999). Various serotyping methods have been used, including immuno-precipitation (Wilkinson and Moody, 1969), enzyme immunoassay (Holm and Hakansson, 1988), coagglutination (Hakansson et al., 1992), counter-immunoelectrophoresis, and capillary precipitation (Triscott and Davies, 1979), latex agglutination (Zuerlein et al., 1991), fluorescence microscopy (Cropp et al., 1974) and inhibition-ELISA (Arakere et al., 1999). These methods are labour-intensive and require high-titered serotype-specific antisera, which are expensive and difficult to make and commercially available for only six serotypes—Ia to V (Arakere et al., 1999). Molecular genotyping methods, such as pulsed-field gel electrophoresis (Rolland et al., 1999), restriction endonuclease analysis (Nagano et al., 1991) are useful for epidemiological studies but do not generally identify serotypes. Consequently, there is a need for a reliable molecular method for GBS serotype identification.

### SUMMARY OF THE INVENTION

We have identified specific regions within the genome of group B streptococci of inter-type sequence heterogeneity that can be used to distinguish different types (including capsular polysaccharide gene serotypes and serosubtypes; protein antigen gene subtypes; and mobile genetic element subtypes). We have shown that molecular methods that detect these sequence heterogeneities can be used to accurately distinguish and type group B streptococci.

Accordingly in a first aspect the present invention provides a method of typing a group B streptococcal bacterium which method comprises analysing the nucleotide sequence of one or more regions within the cpsD, cpsE, cpsF, cpsG, cpsI/M genes of said bacterium, said region(s) comprising one or more nucleotides whose sequence varies between types.

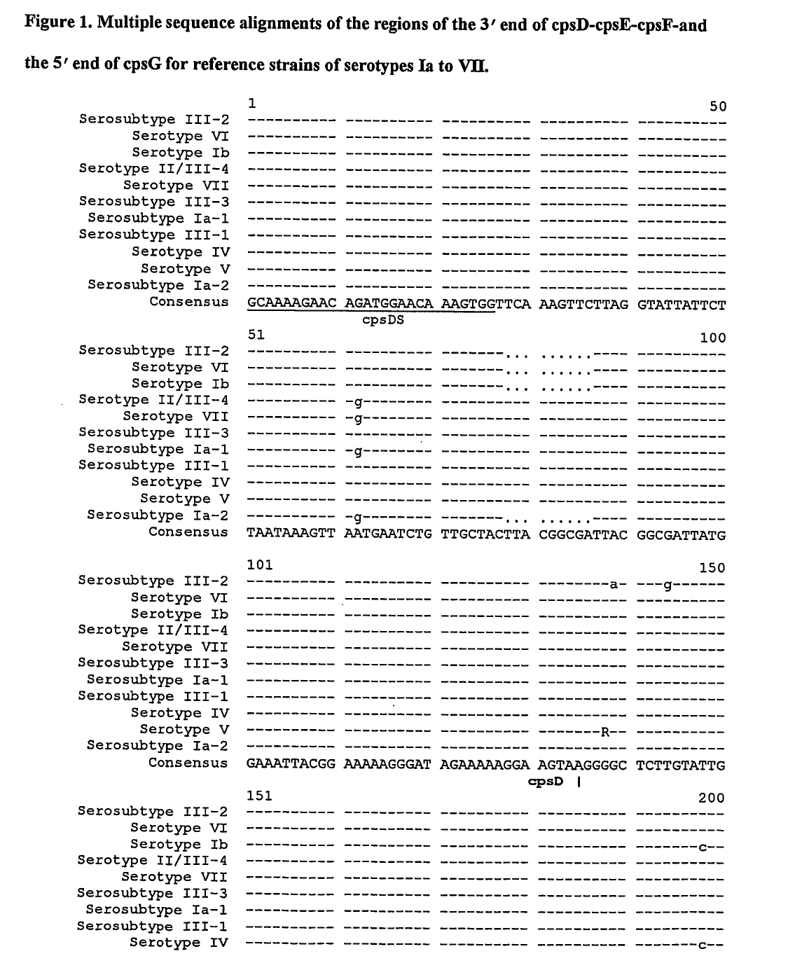

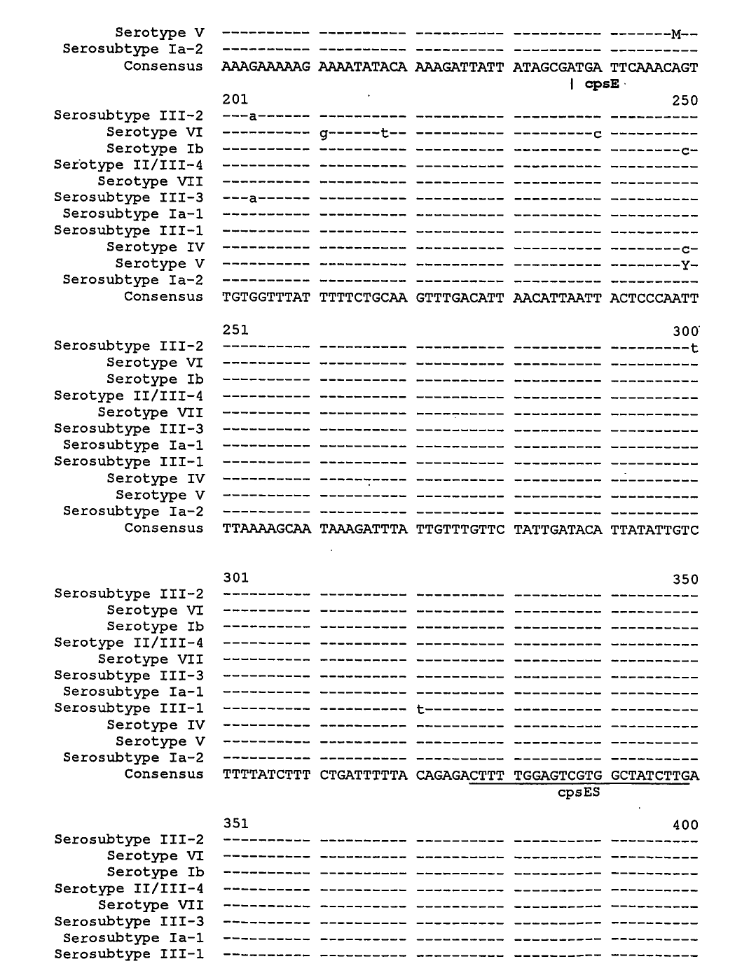

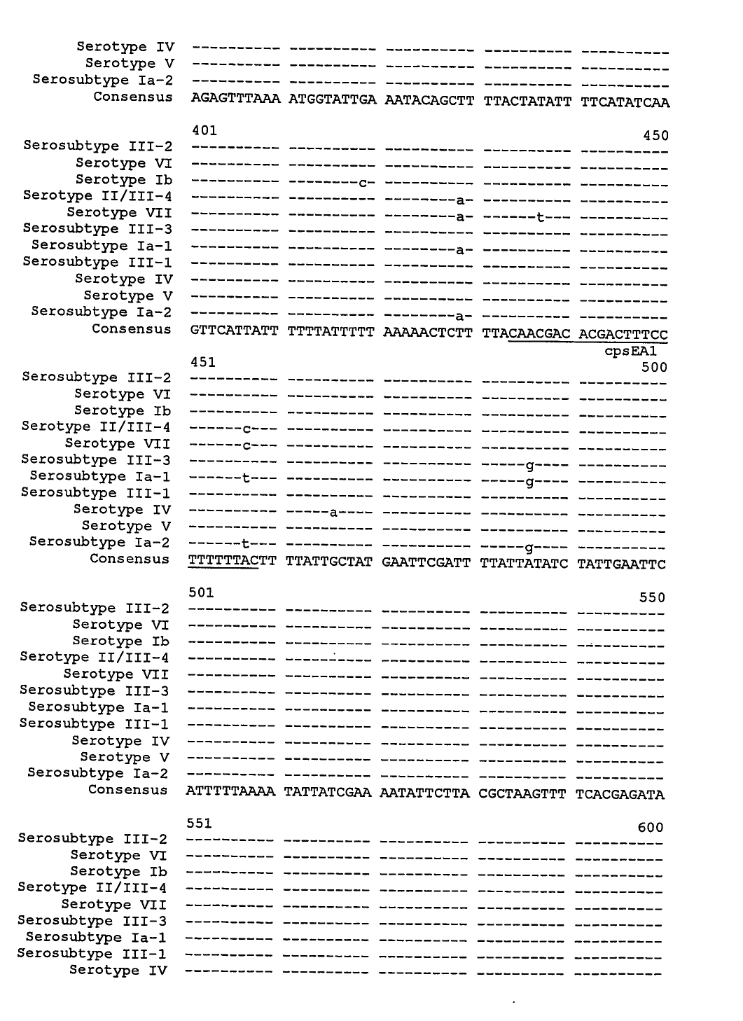

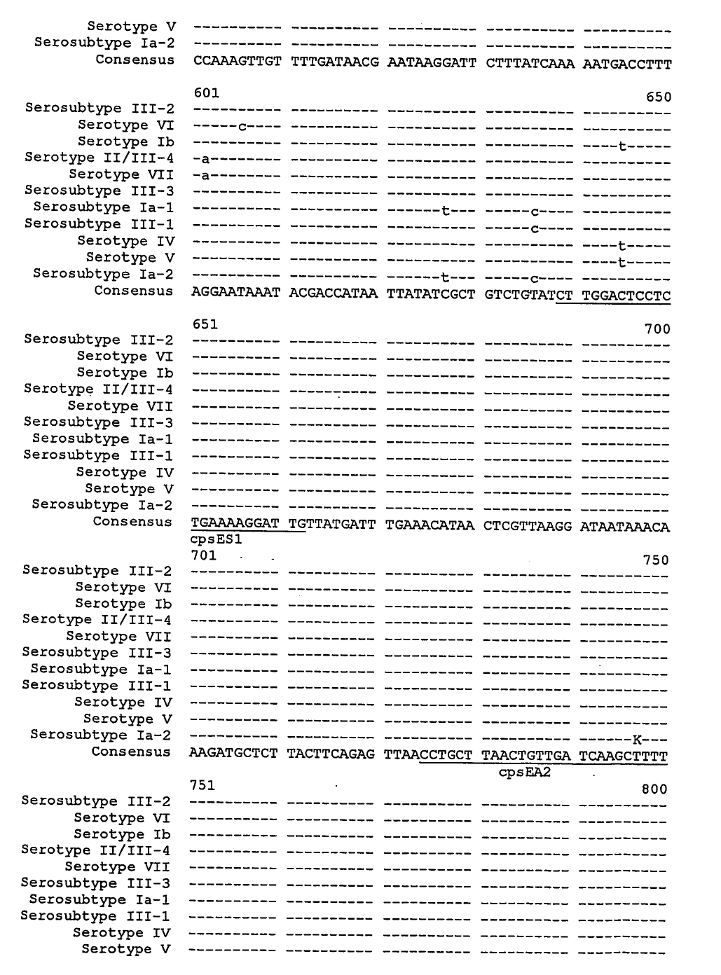

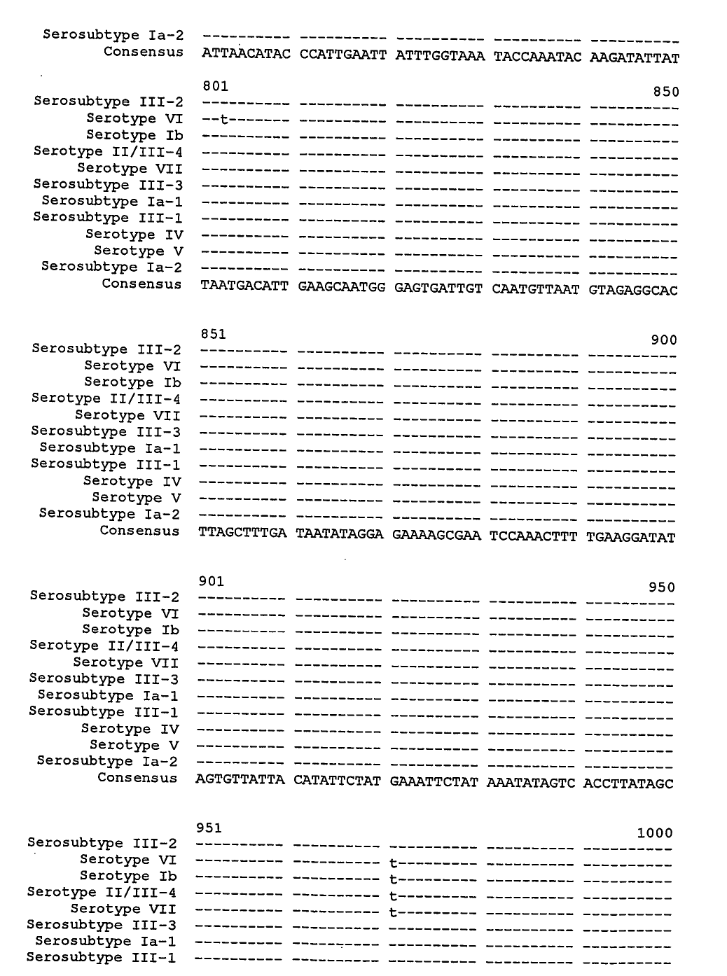

In particular, the nucleotide sequence may be analysed for one or more positions corresponding to positions 62, 78-86, 138, 139, 144, 198, 204, 211, 281, 240, 249, 300, 321, 419, 429, 437, 457, 466, 486, 602, 606, 627, 636, 645, 803, 971, 1026, 1044, 1173, 1194, 1251, 1278, 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

Preferably at least one region is within a sequence delineated by the 3′ 136 bases of the cpsE gene and the 5′ 218 bases of the cpsG gene of the cpsE-cpsF-cspG gene cluster of said group B streptococcal bacterium. In particular, the nucleotide sequence may be analysed for one or more positions corresponding to positions 1413, 1495, 1500, 1501, 1512, 1518, 1527, 1595, 1611, 1620, 1627, 1629, 1655, 1832,1856, 1866, 1871, 1892, 1971, 2026, 2088, 2134, 2187 and 2196 as shown in FIG. 1.

In one embodiment, at least one region is within the cpsI/M genes of said group B streptococcal bacterium.

We have also shown that a number of surface protein antigen genes, including rib, alp2 or alp3 genes, and five mobile genetic elements may be used to molecular subtype GBS. Accordingly, the present invention also provides a method of typing a group B streptococcal bacterium which method comprises determining the presence or absence in the genome of said bacterium of one or more surface protein antigen genes selected from a rib, alp2 or alp3 gene, and/or one or more mobile genetic elements selected from IS861, IS1548, IS1381, ISSa4 and GBSi1. Preferably, such as method is combined with the above methods of the invention.

The nucleotide sequence analysis step may comprise sequencing said one or more regions. Alternatively, or in addition, the nucleotide sequence analysis step may comprises determining whether a polynucleotide obtained from said bacterium selectively hybridises to a polynucleotide probe comprising one or more of the said regions, preferably to one or more of a plurality of polynucleotide probes corresponding to one or more of the said regions.

In a preferred embodiment, where hybridisation to a plurality of probes is used as a means of analysis, the plurality of polynucleotide probes are present as a microarray.

In another embodiment, the nucleotide sequence analysis step comprises an amplification step using one or more primers, at least one of which hybridise specifically to a sequence which differs between types. Typically, primer pairs are used, at least one of which hybridise specifically to a sequence which differs between types. Preferably, said primers are selected from the primers shown in Table 2 and/or Table 6 and/or Table 10.

In a second aspect, the present invention provides a polynucleotide consisting essentially of at least 10 contiguous nucleotides corresponding to a region within a cpsD-cpsE-cpsF-cpsG gene of a group B streptococcal bacterium, said polynucleotide comprising one or more nucleotides which differ between GBS types.

Preferably the nucleotides which differ between GBS types correspond to one or more of positions 62, 78-86, 138, 139, 144, 198, 204, 211, 281, 240, 249, 300, 321, 419, 429, 437, 457, 466, 486, 602, 606, 627, 636, 645, 803, 971, 1026, 1044, 1173, 1194, 1251, 1278, 1413,1495, 1500, 1501, 1512, 1518,1527, 1595, 1611, 1620, 1627, 1629, 1655, 1832, 1856, 1866, 1871, 1892, 1971, 2026, 2088, 2134, 2187 and 2196 as shown in FIG. 1.

The present invention also provides a polynucleotide consisting essentially of at least 10 contiguous nucleotides corresponding to a region within a sequence delineated by the 3′ 136 base pairs of cpsE and the 5′ 218 base pairs of cpsG of the cpsE-cpsF-cspG gene cluster of a group B streptococcal bacterium, said polynucleotide comprising one or more nucleotides which differ between GBS types.

Preferably the nucleotides which differ between group B streptococcal types correspond to one or more of positions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

The present invention also provides a polynucleotide consisting essentially of at least 10 contiguous nucleotides corresponding to a region within a cpsI/M gene of a group B streptococcal bacterium, said polynucleotide comprising one or more nucleotides which differ between group B streptococcal types.

Preferably the polynucleotide is selected from the nucleotide sequences shown in Table 2.

The present invention further provides a polynucleotide consisting essentially of at least 10 contiguous nucleotides corresponding to a region within a rib, alp2 or alp3 gene of a group B streptococcal bacterium, said polynucleotide comprising one or more nucleotides which differ between GBS protein antigen gene subtypes.

Preferably the polynucleotide is selected from the nucleotide sequences shown in Table 6.

The present invention further provides a polynucleotide consisting essentially of at least 10 contiguous nucleotides corresponding to a region within IS861, IS1548, IS1381, ISSa4 and/or GBSi1 of a group B streptococcal bacterium, said polynucleotide comprising one or more nucleotides which differ between GBS mobile genetic element subtypes.

Preferably the polynucleotide is selected from the nucleotide sequences shown in Table 10.

The polynucleotides of the invention may be used in a method of typing, such as serotyping and/or subtyping, a group B streptococcal bacterium.

In a third aspect the present invention provides a composition comprising a plurality of polynucleotides of the second aspect of the invention. The composition may be used in a method of typing, such as serotyping and/or subtyping, a group B streptococcal bacterium.

In a fourth aspect the present invention provides a microarray comprising a plurality of polynucleotides according to the second aspect of the invention. The microarray may be used in a method of typing, such as serotyping and/or subtyping, a group B streptococcal bacterium.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 3^{rd} ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

The molecular typing methods of the present invention rely on detecting the presence in sample of specific polynucleotide sequences in regions of the genome of group B streptococci (GBS) that we have identified as varying between different types.

More specifically, the specific polynucleotide sequences that are to be detected lie within cpsD, cpsE, cpsF, cpsG, cpsI, cpsM, rib, alp2 and/or alp3 genes of GBS as well as mobile genetic elements IS861, IS1548 and IS1381, ISSa4 and GBSi1, preferably the cpsD, cpsE, cpsF, cpsG and/or cpsI/M genes.

Regions of interest within those genes mentioned are regions whose sequence varies between two or more types, i.e. are heterogenous. Heterogeneity may be due to insertions, deletions and/or substitutions between corresponding regions in different types. In the case of rib, alp2 and alp3, heterogeneity typically takes the form of the presence or absence of the entire gene. Similarly for elements IS861, IS1548, IS1381, ISSa4 and GBSi1 heterogeneity typically takes the form of the presence or absence of the entire sequence.

Specific regions of heterogeneity include the following positions within cpsD gene—62 and 78-86; cpsD-cpsE gene spacer—138, 139 and 144; cpsE gene—198, 204, 211, 281, 240, 249, 300, 321, 419, 429, 437, 457, 466, 486, 602, 606, 627, 636, 645, 803, 971, 1026, 1044, 1173, 1194, 1251, 1278, 1413, 1495, 1500, 1501, 1512, 1518 and 1527; cpsF gene—1595, 1611, 1620, 1627, 1629, 1655, 1832, 1856, 1866, 1871, 1892 and 1971; and cpsG gene—2026, 2088, 2134, 2187 and 2196 (numbering corresponds to numbering shown in FIG. 1).

Particularly preferred positions of interest are those that lie within a 790 bp fragment of cpsE-cps-F-cpsG (which consists of approximately the 3′ 136 bases of cpsE to the 5′ 218 bases of cpsG), namely positions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

Another region of heterogeneity is position 62 of cpsD and a repetitive sequence (TRACGGCGA) found at positions 78 to 86 of cpsD in some but not all GBS serotypes.

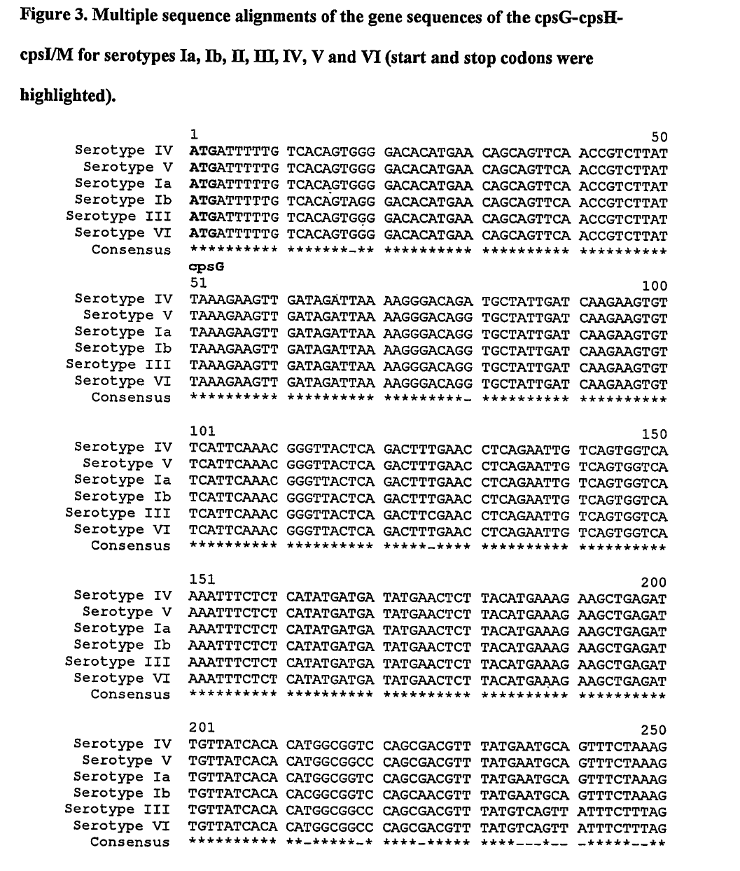

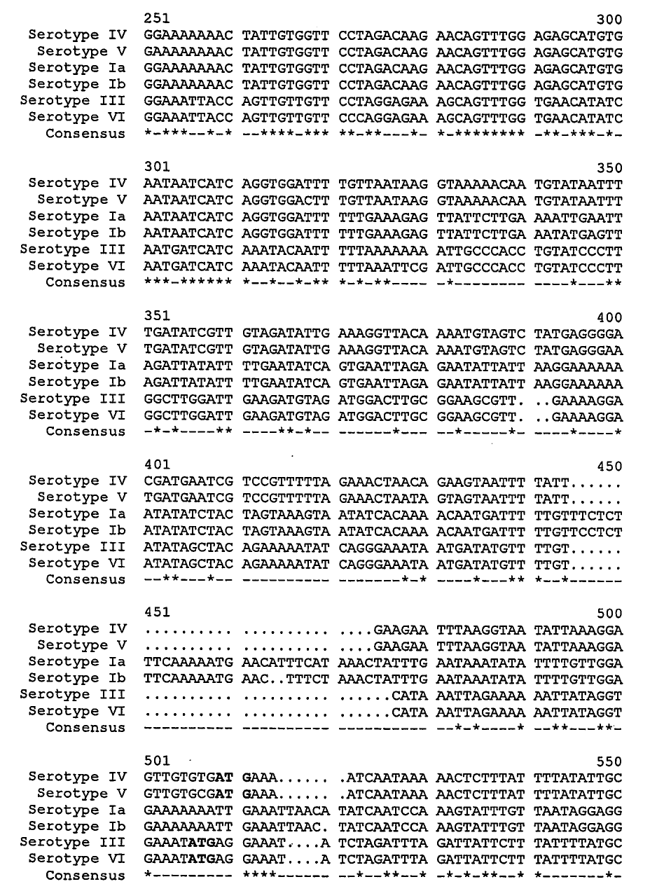

Specific regions of heterogeneity also include a number of positions within the cpsI/M gene as shown in the sequence alignment depicted in FIG. 3.

These regions of heterogeneity may be analysed using a variety of means including sequencing, PCR and binding of labelled probes.

In the case of sequencing to identify serotype, the sequencing primers are selected such that they hybridise specifically to a region within or near to a region within which a region of heterogeneity is present. The primers need not be specific to particular serotypes since the actual sequence information obtained during the sequencing process which is used to assign molecular serotype. Thus the primers may hybridise specifically to all GBS serotypes (at least serotypes Ia to VII), or to specific serotypes.

Preferred primers anneal within 100, 50 or 20 contigous nucleotides of a heterogeneous position within the 790 bp region of cpsE-cpsF-cpsG shown in FIG. 1. Examples of suitable sequencing primers are shown in Table 2 (cpsES3, cpsFA, cpsFS, cpsGA and cpsGA1).

PCR and other specific hybridisation-based serotyping methods will typically involve the use of nucleotide primers/probes which bind specifically to a region of the genome of a GBS serotype which includes a nucleotide which varies between two or more serotypes. Thus the primers/probes may comprise a sequence which is complementary to one of such regions. Where positions of heterogeneity are close together (e.g. positions 198, 204, 211 and 218 of cpsE), it may be desirable to use a primer/probe which hybridises specifically to a region of the GBS genome that comprises two or more positions of heterogeneity. Thus for example, a primer/probe may be designed that is complementary to nucleotides 195 to 220 of cpsE. Such primers/probes are likely to have improved specificity and reduce the likelihood of false positives.

PCR-based methods of detection may rely upon the use of primer pairs, at least one of which binds specifically to a region of interest in one or more, but not all, serotypes. Unless both primers bind, no PCR product will be obtained. Consequently, the presence or absence of a specific PCR product may be used to determine the presence of a sequence indicative of specific GBS serotypes. However, as mentioned, only one primer need correspond to a region of heterogeneity in the genes of interest (such as the cpsD, cpsE, cpsF, cpsG, cpsI and/or cpsM genes). The other primer may bind to a conserved or heterogenous region within said gene or even a region within another part of the GBS genome, such as the cpsH gene, whether said region is conserved or heterogeneous between serotypes. Thus, for example, a combination of a primer (cpsGS) which binds to a region of the cpsG gene including positions 2172 to 2210, and a primer which binds to a region of cpsH gene which is heterogeneous (lacpsHA1, IIIcpsHA), may be used as the basis of distinguishing serotypes (Ia and III).

Further, a primer which binds to a region of cpsI which is heterogeneous may be combined with a primer which binds to a region of cpsG which is constant. An example of such as primer pair is primer pair VIcpsIA, and cpsGS1, which give rise to a PCR product of 1517 bp and GBS serotype VI specific.

Alternatively, primers that bind to conserved regions of the GBS genome but which flank a region whose length varies between serotypes may be used. In this case, a PCR product will always be obtained when GBS bacteria are present but the size of the PCR product varies between serotypes.

Furthermore, a combination of specific binding of one or both primers and variations in the length of PCR primer may be used as a means of identifying particular molecular serotypes.

Examples of specific primers/probes which target the cpsD, cpsE, cpsF, cpsG, cpsI or cpsM genes include the following:

| | |
|---|---|
| cpsDS | GCA AAA GAA CAG ATG GAA CAA AGT GG |
| cpsES | CTT TTG GAG TCG TGG CTA TCT TG |
| cpsEA1 | GA/T/GA AAA AAG GAA AGT CGT GTC G/ATT G |
| cpsES1 | CTT GGA C/TTC CTC TGA AAA GGA TTG |
| cpsEA2 | AAA A/CGC TTG ATC AAC AGT TAA GCA GG |
| cpsES2 | GAT GGT/C GGA CCG GCT ATC TTT TCT C |
| cpsEA3 | CTT AAT TTG TTC TGC ATC TAC TCG C |
| cpsES3 | GTT AGA TGT TCA ATA TAT CAA TGA ATG GTC TAT TTG GTC AG |
| cpsEFA | CCT TTC AAA CCT TAC CTT TAC TTA GC |
| cpsFS | CAT CTG GTG CCG CTG TAG CAG TAC CAT T |
| cpsFA | GTC GAA AAC CTC TAT A/GT A AAC/T GGT CTT ACA A/GCC AAA |
| | TAA CTT ACC |
| cpsGA | AAG/C AGT TCA TAT CAT CAT ATG AGA G |
| cpsGA1 | CCG CCA/G TGT GTG ATA ACA ATC TCA GCT TC |
| cpsGS | ATG ATG ATA TGA ACT CTT ACA TGA AAG AAG CTG AGA TTG |
| cpsGS1 | GAA CTC TTA CAT GAA AGA AGC TGA GAT TGT TAT CAC AC |
| IbcpsIA | CTA TCA ATG AAT GAG TCT GTT GTA GGA CGG ATT GCA CG |
| IbcpsIS | GAT AAT AGT GGA GAA ATT TGT GAT AAT TTA TCT CAA AAA |
| | GAC G |
| IbcpsIA1 | CCT GAT TCA TTG CAG AAG TCT TTA CGA TGC GAT AGG TG |
| IVcpsMA | GGG TCA ATT GTA TCG TCG CTG TCA ACA AAA CCA ATC AAA TC |
| VcpsMA | CCC CCC ATA AGT ATA AAT AAT ATC CAA TCT TGC ATA GTC AG |
| VIcpsIA | GAA GCA AAG ATT CTA CAC AGT TCT CAA TCA CTA ACT CCG |
| cpsIA | GTA TAA CTT CTA TCA ATG GAT GAG TCT GTT GTA GTA CGG |

The primer designations correspond to those given in Table 2.

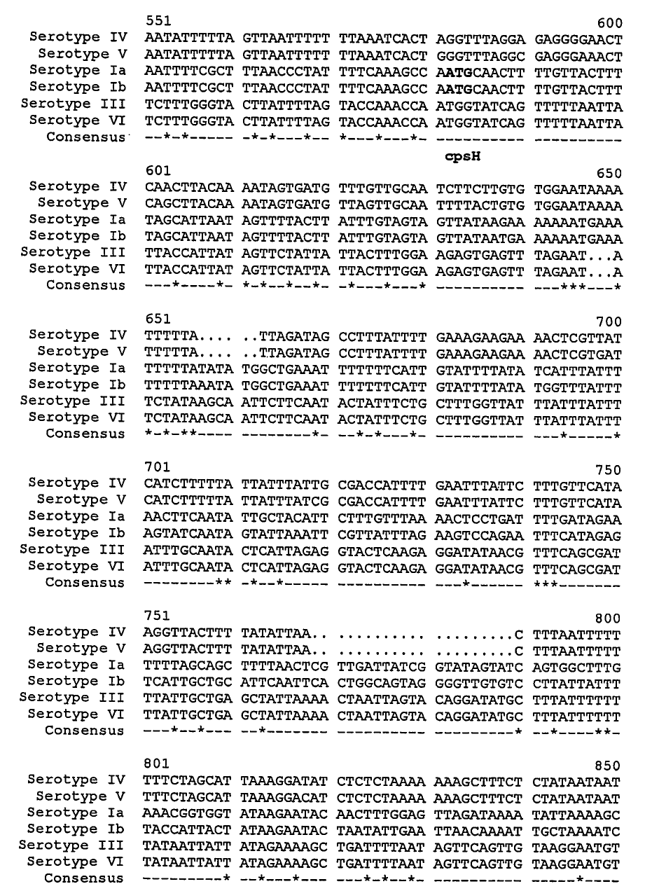

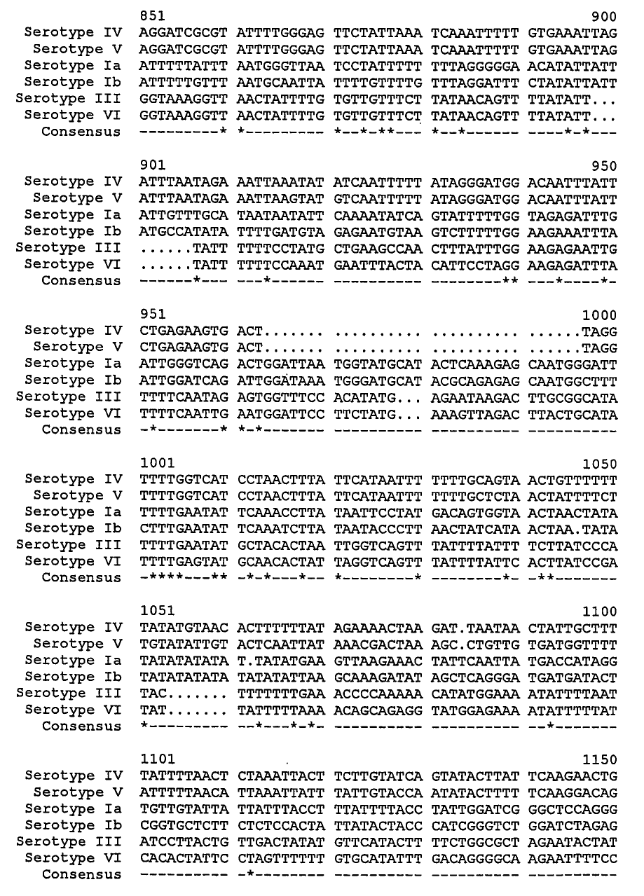

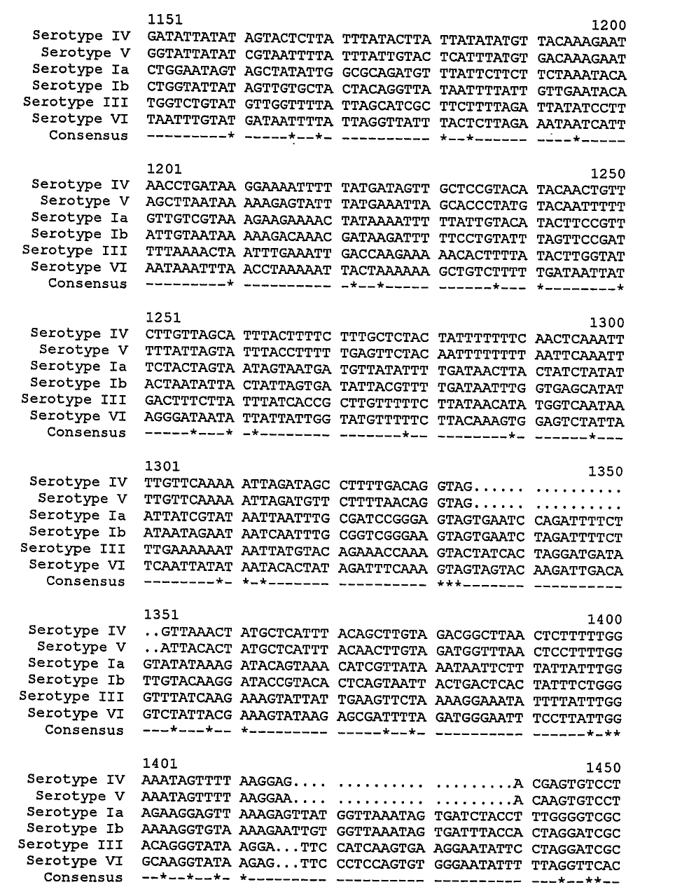

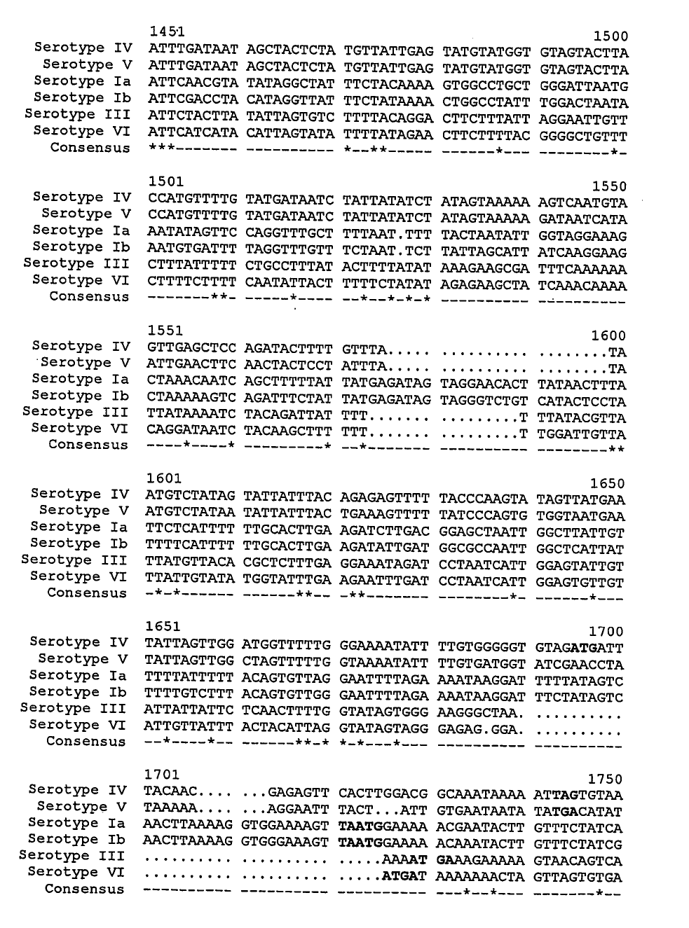

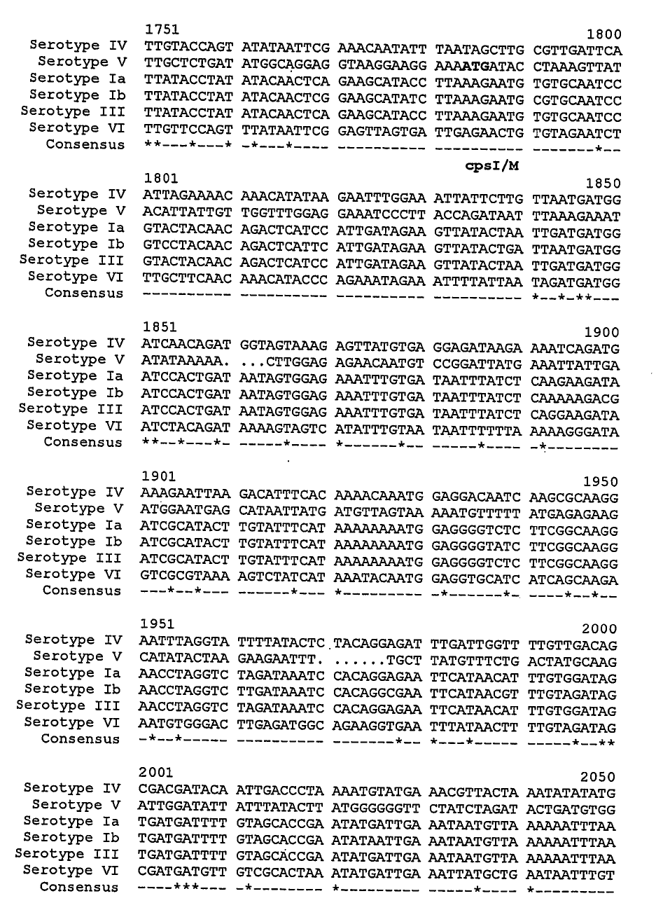

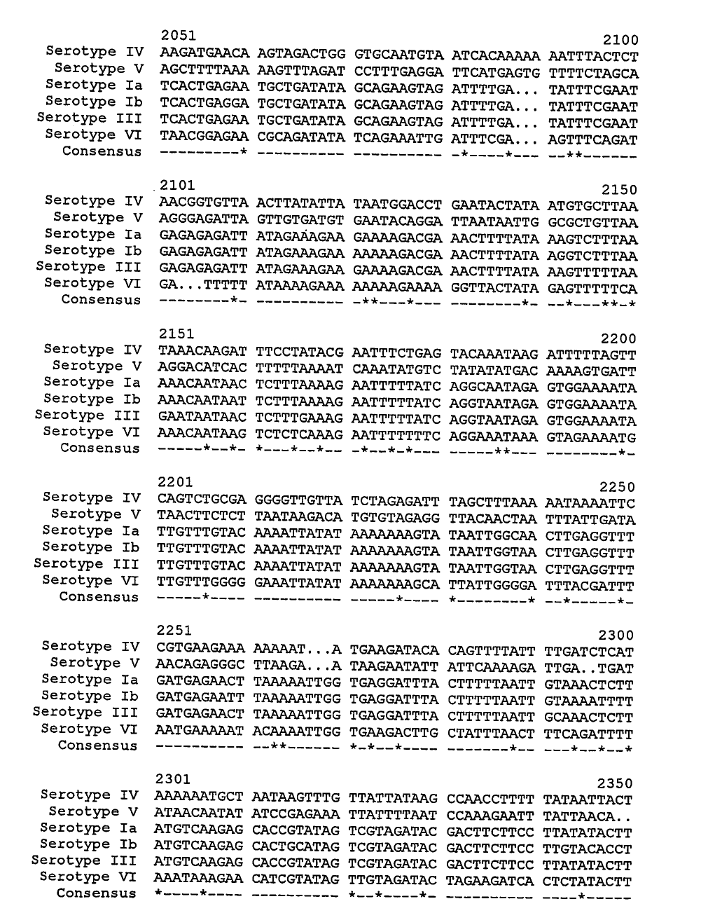

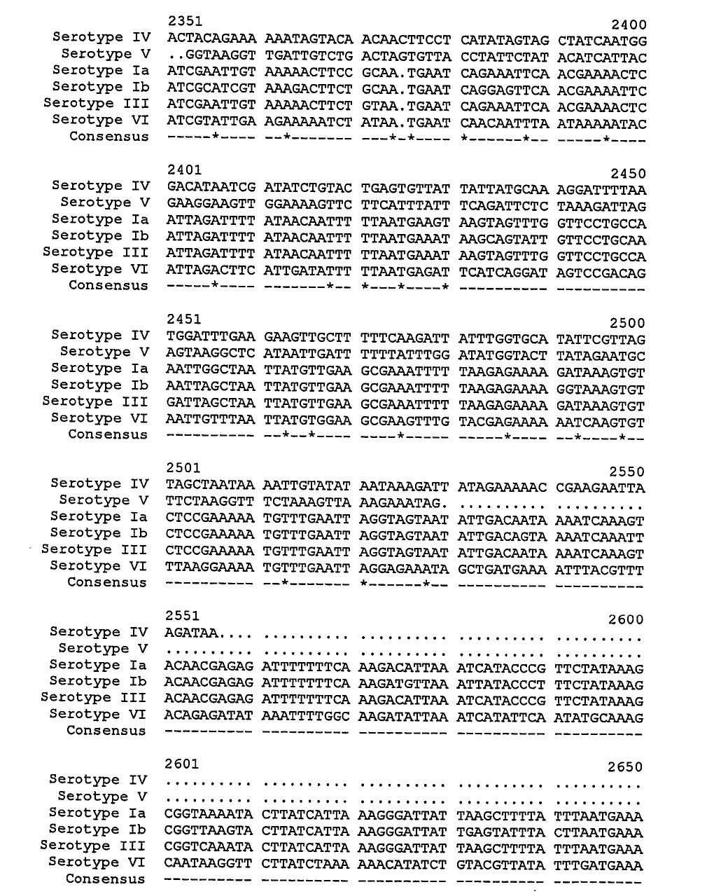

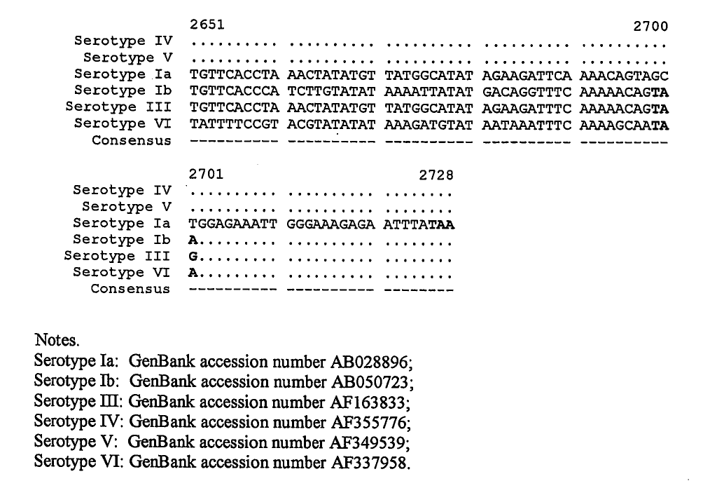

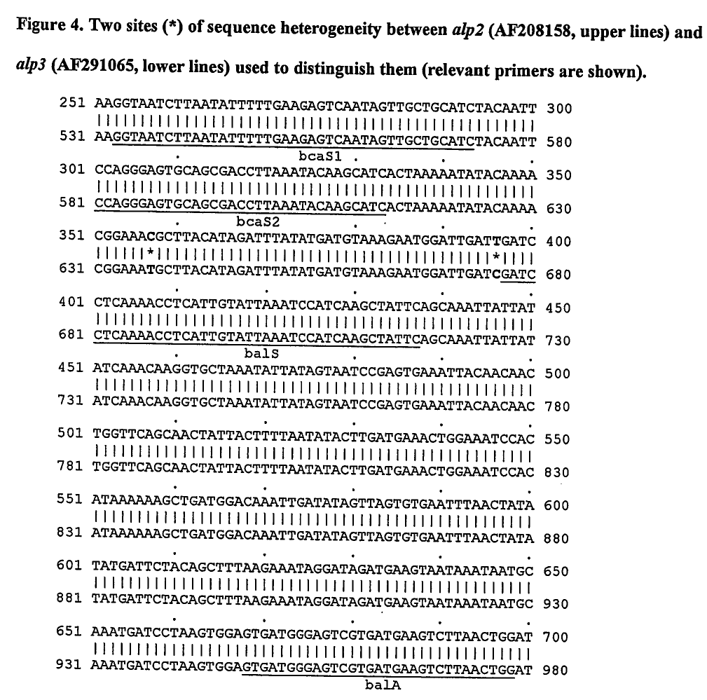

In relation to the alp2, alp3 and rib surface protein antigen genes, heterogeneity and protein antigen gene subtype is assessed more at the level of whether a group B streptococcal bacterium contains the gene or not. Our results show that the specific combination of surface proteins genes present in a GBS genome is indicative of serotype/serosubtypes (see Table 9). Consequently, primers/probes suitable for use in the methods of the present invention are those that are specific for the particular genes. Thus probes/primers that are specific for alp2 or alp3 or rib are preferred. FIG. 4 shows an alignment of alp2 and alp3 that was used to design primers specific for alp2 or specific for alp3.

Examples of specific primers/probes which target the alp2, alp3 and rib genes include the following:

| | |
|---|---|
| bcaS1 | GGT AAT CTT AAT ATT TTT GAA GAG TCA ATA GTT GCT GCA TCT |
| | AC |
| bcaS2 | CCAGGGA GTG CAG CGA CCT TAA ATA CAA GCA TC |
| baIS | GAT CCT CAA AAC CTC ATT GTA TTA AAT CCA TCA AGC TAT TC |
| baIA | CCA GTT AAG ACT TCA TCA CGA CTC CCA TCA C |
| baI23S1 | CAG ACT GTT AAA GTG GAT GAA GAT ATT ACC TTT ACG G |
| baI23S2 | CTT AAA GCT AAG TAT GAA AAT GAT ATC ATT GGA GCT CGT G |
| baI2S | CTT CCG CCA GAT AAA ATT AAG |
| baI2A | CTG TTG ACT TAT CTG GAT AGG TC |
| baI2A1 | CGT GTT GTT CAA CAG TCC TAT GCT TAG CCT CTG GTG |
| baI2A2 | GGT ATC TGG TTT ATG ACC ATT TTT CCA GTT ATA CG |
| baI3S | GTT CTT CCG CTT AAG GAT AG |
| baI3A | GAC CGT TTG GTC CTT ACC TTT TGG TTC GTT GCT ATC C |
| ribS2 | GAAGTAATTTCAG GAA GTG CTG TTA CGT TAA ACA CAA ATA TG |
| ribA1 | GAA GGT TGT GTG AAA TAA TTG CCG CCT TGC CTA ATG |
| ribA2 | AAT ACT AGC TGC ACC AAC AGT AGT CAA TTC AGA AGG |

The primer designations correspond to those given in Table 6.

In relation to the IS861, IS1548, IS1381, ISSa4 and GBSi1, heterogeneity and subtype is assessed more at the level of whether a group B streptococcal bacterium contains the element or not. The number of elements may also be assessed. Our results show that the specific combination of mobile elements present in a GBS genome is indicative of serotype/serosubtype (see Table 12). Consequently, primers/probes suitable for use in the methods of the present invention are those that are specific for the particular mobile genetic elements. Thus probes/primers that are specific for IS861, IS1548, IS1381, ISSa4 and GBSi1 are preferred.

Examples of specific primers/probes which target IS861, IS1548, IS1381, ISSa4 and GBSi1 include the following:

| | |
|---|---|
| IS861S | GAG AAA ACA AGA GGG AGA CCG AGT AAA ATG GGA CG |
| IS861A1 | CAC GAT TTC GCA GTT CTA AAT AAA TCC GAC GAT AGC C |
| IS861A2 | CAA ACT CCG TCA CAT CGG TAT AGC ACT TCT CAT AGG |
| IS1548S | CTA TTG ATG ATT GCG CAG TTG AAT TGG ATA GTC GTC |
| IS1548S1 | GTT TGG GAC AGG TAG CGG TTG AGG AGA AAA GTA ATG |
| IS1548A1 | CAT TAC TTT TCT CCT CAA CCG CTA CCT GTC CCA AAC |
| IS1548A2 | CCC AAT ACC ACG TAA CTT ATG CCA TTT G |
| IS1548A3 | CGT GTT ACG AGT CAT CCC AAT ACC ACG TAA CTT ATG CC |
| IS1381S1 | CTT ATG AAC AAA TTG CGG CTG ATT TTG GCA TTC ACG |
| IS1381S2 | GGC TCA GGC GAT TGT CAC AAG CCA AGG GAG |
| IS1381A | CTA AAA TCC TAG TTC ACG GTT GAT CAT TCC AGC |
| ISSa4S | CGT ATC TGT CAC TTA TTT CCC TGC GGG TGT CTC C |
| ISSa4A1 | GCC GAT GTC ACA ACA TAG TTC AGG ATA TAG CCA G |
| ISSa4A2 | CGT AAA GGA GTC CAA AGA TGA TAG CCT TTT TGA ACC |
| GBSi1S1 | CAT CTC GGA ACA ATA TGC TCG AAG CTT ACA AGC AAG TG |
| GBSi1S2 | GGG GTC ACT ATC GAG CAG ATG GAT GAC TAT CTT CAC |
| GBSi1A1 | AAT GGC TGT TTC GCA GGA GCG ATT GGG TCT GAA CC |
| GBSi1A2 | CCA GGG ACA TCA ATC TGT CTT GCG GAA CAG TAT CG |

Preferably, the primers/probes comprise at least 10, 15 or 20 nucleotides. Typically, primers/probes consist of fewer than 100, 50 or 30 nucleotides. Primers/probes are generally polynucleotides comprising deoxynucleotides. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Primers/probes may be labelled with any suitable detectable label such as radioactive atoms, fluorescent molecules or biotin.

In one embodiment, primers/probes have a high melting temperature of >70° C. so that they may be used in rapid cycle PCR.

Compositions comprising a plurality of nucleotides that are used to analyse one or more regions within the cpsD, cpsE, cpsF, cpsG, or cpsI/M genes may also further comprise nucleotides that may be used to analyse one or more regions within the cpsH gene. Suitable nucleotides are described in the Examples, although a person skilled in the art could design other suitable sequences based on the sequence alignment shown in FIG. 3.

Further, compositions comprising a plurality of nucleotides that are used to analyse one or more regions within alp2, alp3 or rib genes may also further comprise nucleotides that may be used to analyse one or more regions within the C alpha (boa) and C beta (bac) genes (C beta gene also known as bag).

A variety of techniques may be used to analyse one or more regions within the genome of a bacterium of interest. Typically, a sample of interest, which is suspected of containing GBS bacteria is treated, using standard techniques to obtain genomic DNA from any microorganisms present in the sample. It may be desirable for a number of subsequent detection steps to use nucleic acid preparation techniques that result in substantial fragmentation of the genomic DNA. The sample may be from a bacterial culture or a clinical sample from a patient, typically a human patient. Clinical samples may be cultured to produce a bacterial culture. However, it is also possible to test clinical samples directly with a culturing step.

The genomic DNA is then subjected to one or more analysis steps which may include sequencing, enzymatic amplification and/or hybridisation. These general techniques of DNA analysis are known in the art and are discussed in detail in, for example, Sambrook et al. 2001 and Ausubel et al. 1999 supra.

Serotyping may involve a one or more steps. For example, it may be desirable to carry out an initial step of determining whether there are nucleotide sequences present in the sample which are conserved between GBS seroptypes but not found in any other organism. This may be achieved by using PCR primers that detect any (but only) GBS bacteria (e.g. using primer pairs Sag59/Sag190 and/or DSF2/DSR1—see Tables 2 and 3).

Molecular serotyping for specific GBS serotypes can then be performed by detecting the presence of one or more regions of heterogeneity in the regions of interest using any suitable technique such as sequencing, enzymatic amplification and/or hybridisation based on the probes/primers discussed above.

A particularly preferred detection technique is PCR, such as rapid cycle PCR (Kong et al., 2000).

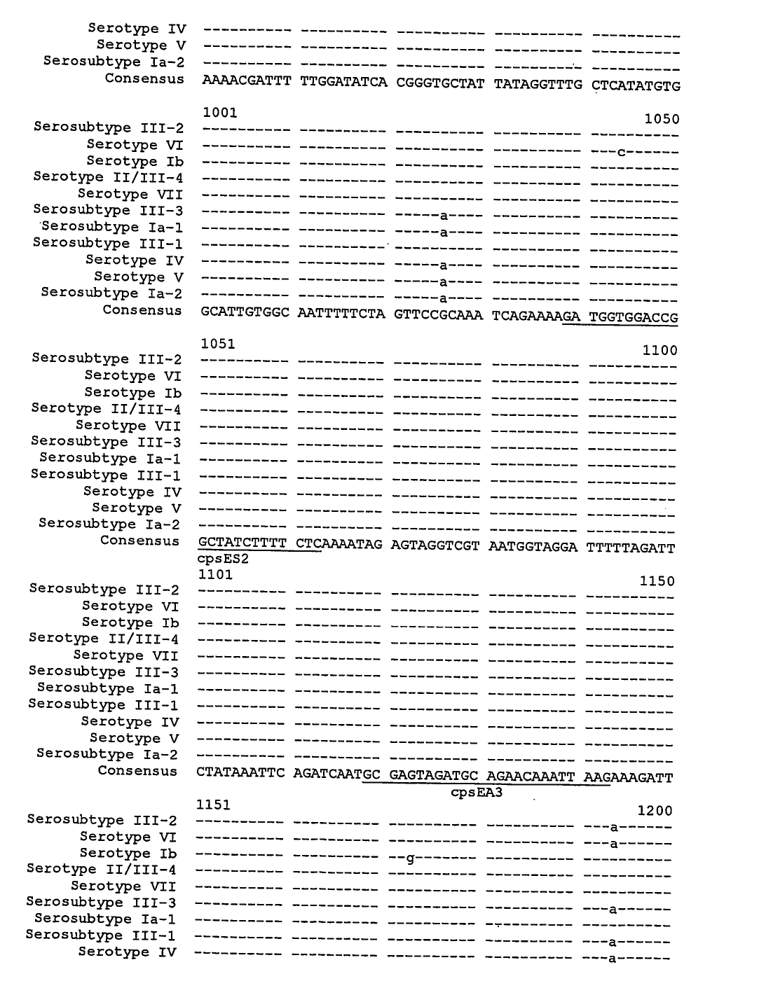

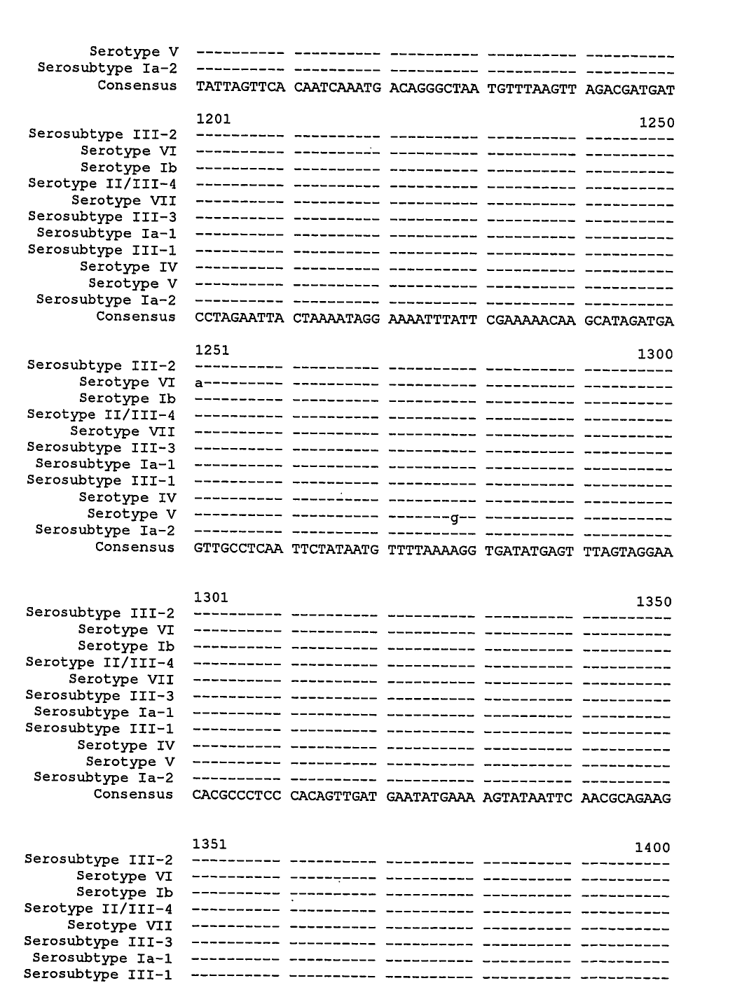

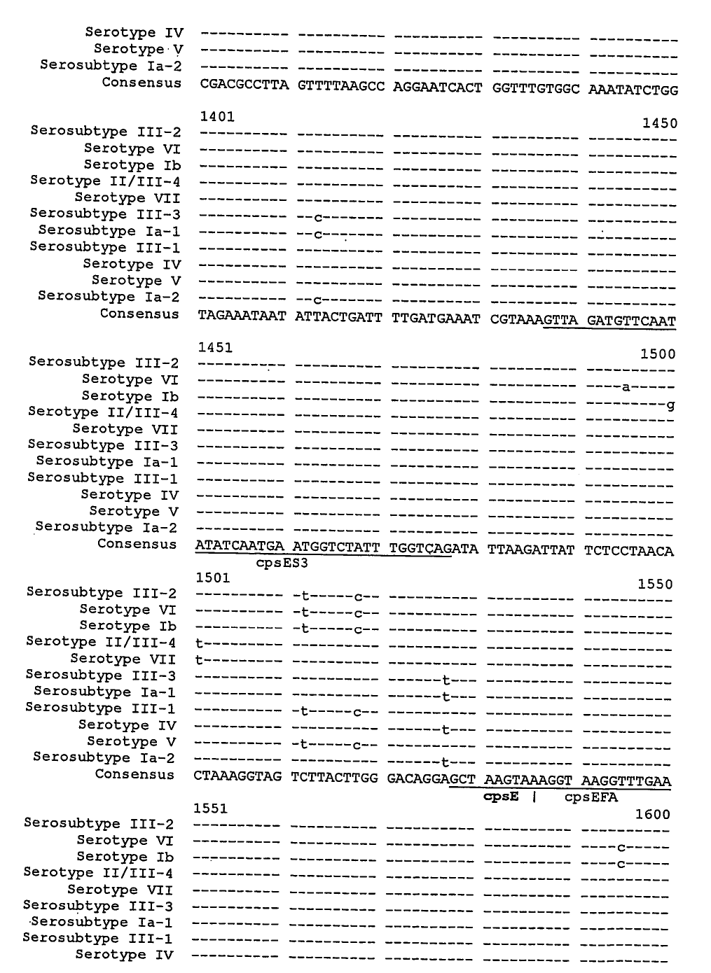

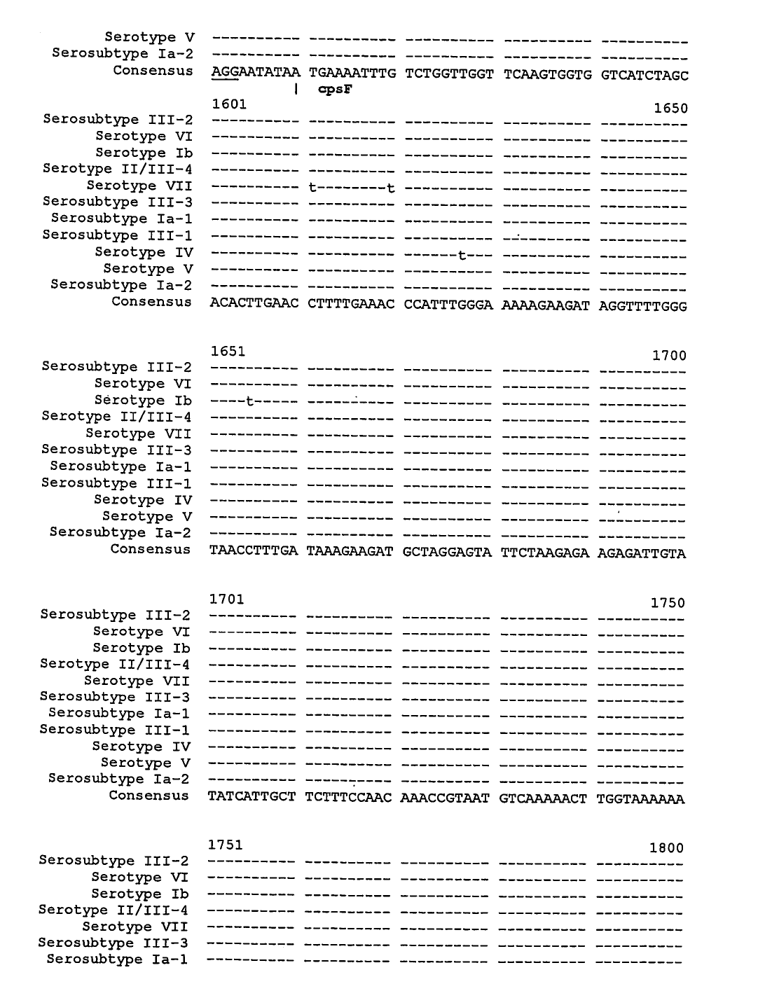

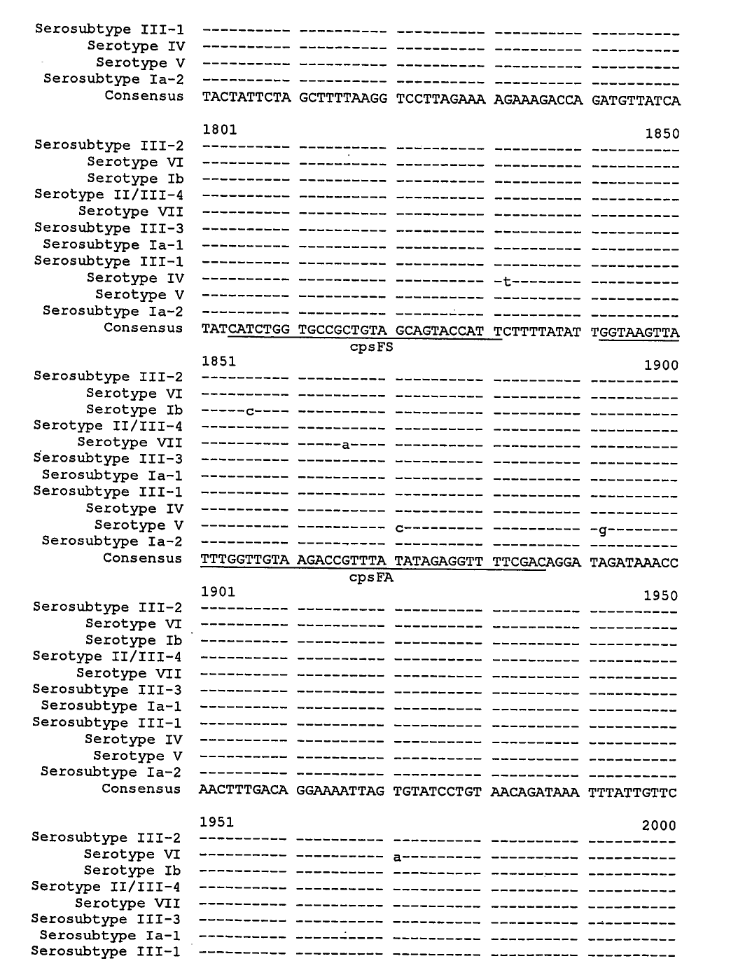

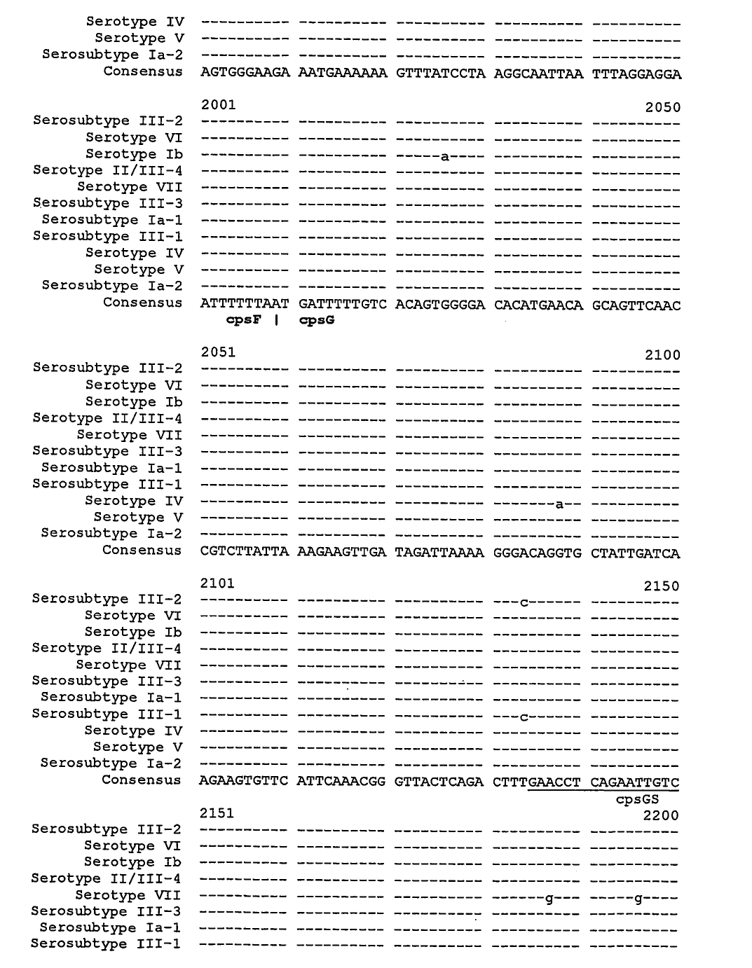

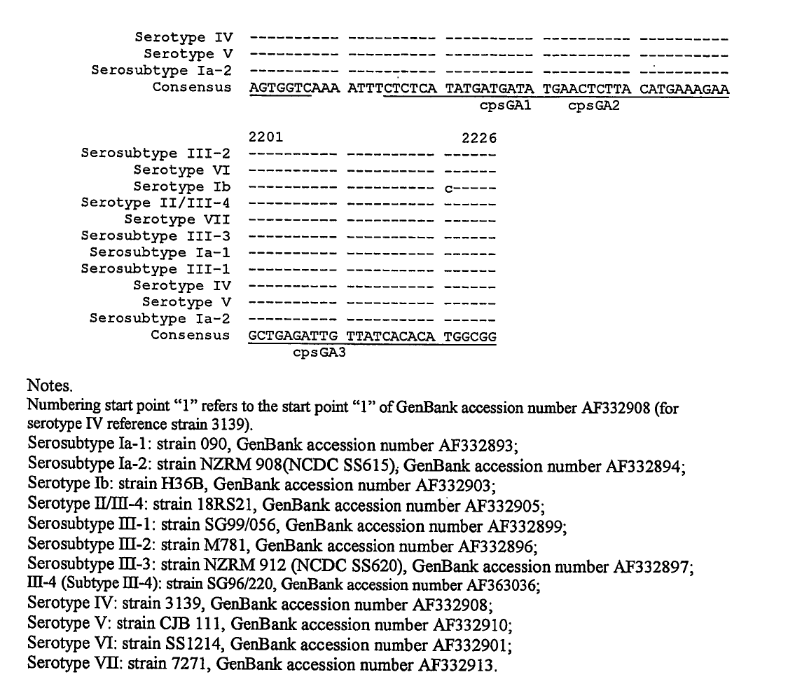

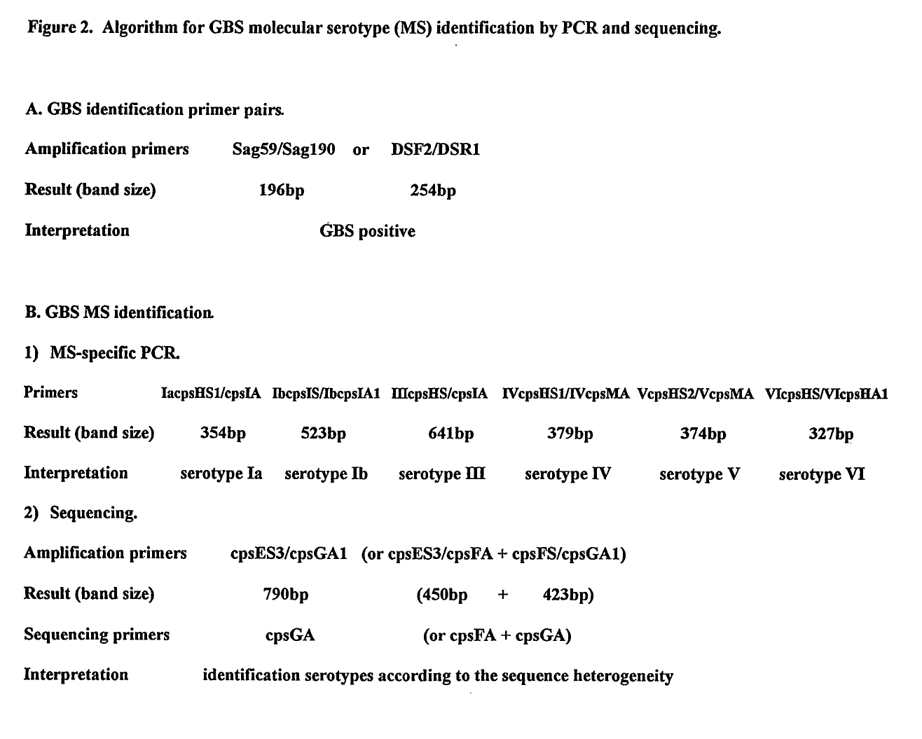

An example of a multi-step serotyping strategy (algorithm) is shown in FIG. 2. However, a variety of other strategies are envisaged and can be designed by the skilled person using the sequence heterogeneity information presented herein. In particular, it is preferred that the serotyping procedure comprise at least one analysis step based on analysing one or regions of the cpsD, cpsE, cpsF, cpsG and/or cpsI/M genes. This analysis may optionally be combined with an analysis of one or more regions within the cpsH gene. Similar techniques may be used to analyse the cpsH gene regions and suitable primer sequences and methods are also described in the Examples.

Analysis of the presence of absence of the alp2, alp3 and/or rib genes may optionally be combined with an analysis of the presence or absence of C alpha (bca gene), C beta (bac) gene sequences as is described in the Examples. Similar techniques may be used to analyse these regions and suitable primer sequences and PCR methods are also described in the Examples.

Furthermore, analysis of the presence of absence of the alp2, alp3 and/or rib genes (and optionally the bca and bac genes) may be combined with an analysis of the presence or absence of mobile genetic elements.

Thus a typing strategy may involve an analysis of cps genes, surface protein genes and/or mobile genetic elements in various combinations to provide more serosubtyping and subtyping information.

Analysis of GBS genomic sequences using the above techniques may take place in solution followed by standard resolution using methods such as gel electrophoresis. However in a preferred aspect of the invention, the primers/probes are immobilised onto a solid substrate to form arrays.

The polynucleotide probes are typically immobilised onto or in discrete regions of a solid substrate. The substrate may be porous to allow immobilisation within the substrate or substantially non-porous, in which case the probes are typically immobilised on the surface of the substrate. Examples of suitable solid substrates include flat glass (such as borosilicate glass), silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal.

Preferably, the solid substrate is generally a material having a rigid or semi-rigid surface. In preferred embodiments, at least one surface of the substrate will be substantially flat, although in some embodiments it may be desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm⁻².

The solid substrate is conveniently divided up into sections. This may be achieved by techniques such as photoetching, or by the application of hydrophobic inks, for example teflon-based inks (Cel-line, USA). Discrete positions, in which each different probes are located may have any convenient shape, e.g., circular, rectangular, elliptical, wedge-shaped, etc.

Attachment of the library sequences to the substrate may be by covalent or non-covalent means. The library sequences may be attached to the substrate via a layer of molecules to which the library sequences bind. For example, the probes may be labelled with biotin and the substrate coated with avidin and/or streptavidin. A convenient feature of using biotinylated probes is that the efficiency of coupling to the solid substrate can be determined easily. Since the polynucleotide probes may bind only poorly to some solid substrates, it is often necessary to provide a chemical interface between the solid substrate (such as in the case of glass) and the probes. Thus, the surface of the substrate may be prepared by, for example, coating with a chemical that increases or decreases the hydrophobicity or coating with a chemical that allows covalent linkage of the polynucleotide probes. Some chemical coatings may both alter the hydrophobicity and allow covalent linkage. Hydrophobicity on a solid substrate may readily be increased by silane treatment or other treatments known in the art. Examples of suitable chemical coatings include polylysine and poly(ethyleneimine). Further details of methods for the attachment of are provided in U.S. Pat. No. 6,248,521. Methods for immobilizing nucleic acids by introduction of various functional groups to the molecules are also described in Bischoff et al., 1987 (Anal. Biochem., 164:336-3440 and Kremsky et al, 1987 (Nucl. Acids Res. 15:2891-2910).

Techniques for producing immobilised arrays of nucleic acid molecules have been described in the art A useful review is provided in Schena et al., 1998, TibTech 16: 301-306, which also gives references for the techniques described therein.

Microarray-manufacturing technologies fall into two main categories—synthesis and delivery. In the synthesis approaches, microarrays are prepared in a stepwise fashion by the in situ synthesis of nucleic acids from biochemical building blocks. With each round of synthesis, nucleotides are added to growing chains until the desired length is achieved. A number of prior art methods describe how to synthesise single-stranded nucleic acid molecule libraries in situ, using for example masking techniques (photolithography) to build up various permutations of sequences at the various discrete positions on the solid substrate. U.S. Pat. No. 5,837,832 describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

The delivery technologies, by contrast, use the exogenous deposition of preprepared biochemical substances for chip fabrication. For example, DNA may also be printed directly onto the substrate using for example robotic devices equipped with either pins (mechanical microspotting) or piezo electric devices (ink jetting). In mechanical microspotting, a biochemical sample is loaded into a spotting pin by capillary action, and a small volume is transferred to a solid surface by physical contact between the pin and the solid substrate. After the first spotting cycle, the pin is washed and a second sample is loaded and deposited to an adjacent address. Robotic control systems and multiplexed printheads allow automated microarray fabrication. Ink jetting involves loading a biochemical sample, such as a polynucleotide into a miniature nozzle equipped with a piezoelectric fitting and an electrical current is used to expel a precise amount of liquid from the jet onto the substrate. After the first jetting step, the jet is washed and a second sample is loaded and deposited to an adjacent address. A repeated series of cycles with multiple jets enables rapid microarray production.

In one embodiment, the microarray is a high density array, comprising greater than about 50, preferably greater than about 100 or 200 different nucleic acid probes. Such high density probes comprise a probe density of greater than about 50, preferably greater than about 500, more preferably greater than about 1,000, most preferably greater than about 2,000 different nucleic acid probes per cm². The array may further comprise mismatch control probes and/or reference probes (such as positive controls).

Microarrays of the invention will typically comprise a plurality of primers/probes as described above. The primers/probes may be grouped on the array in any order. However, it may be desirable to group primers/probes according to types (capsular polysaccharide gene serotypes, serosubtypes; protein antigen gene subtypes; mobile genelic elements subtypes), or groups of types (capsular polysaccharide gene serotypes, serosubtypes; protein antigen gene subtypes; mobile genelic elements subtypes) for which they are specific. Such grouping may be arranged such that the resulting patterns are easily susceptible to pattern recognition by computer software.

Elements in an array may contain only one type of probe/primer or a number of different probes/primers.

Detection of binding of GBS genomic DNA to immobilised probes/primers may be performed using a number of techniques. For example, the immobilised probes which are specific to a number of types (capsular polysaccharide gene serotypes, serosubtypes; protein antigen gene subtypes; mobile genelic elements subtypes), may function as capture probes. Following binding of the genomic DNA to the array, the array is washed and incubated with one or more labelled detection probes which hybridise specifically to regions of the GBS genome which are conserved. The binding of these detection probes may then be determined by detecting the presence of the label. For example, the label may be a fluorescent label and the array may be placed in an X-Y reader under a charge-coupled device (CCD) camera.

Other techniques include labelling the genomic DNA prior to contact with the array (using nick-translation and labelled dNTPs for example). Binding of the genomic DNA can then be detected directly.

It is also possible to employ a single PCR amplification step using labelled dNTPs. In this embodiment, the genomic DNA fragment binds to a first primer present in the array. The addition of polymerase, dNTPs, including some labelled dNTPs and a second primer results in synthesis of a PCR product incorporating labelled nucleotides. The labelled PCR fragment captured on the plate may then be detected.

A number of available detection techniques do not require labels but instead rely on changes in mass upon ligand binding (e.g. surface plasmon resonance—SPR). The principles of SPR and the types of solid substrates required for use in SPR (e.g. BIACore chips) are described in Ausubel et al., 1999, supra.

C. Uses

As discussed above, group B streptococcus (GBS)—Streptococcus agalactiae—is the commonest cause of neonatal and obstetric sepsis and an increasingly important cause of septicaemia in the elderly and immunocompromised patients. Thus, the detection methods, probes/primer and microarrays of the invention may be used in the diagnosis of GBS infections in pregnant women, elderly and/or immunocompromised patients. The PCR and microarray techniques described herein may be of particular use in routine antenatal screening of pregnant women as well as in diagnosing infections in pregnant women given the increased accuracy and sensitivity compared to conventional identification and serotyping. These methods are also likely to give faster results since it will not generally be necessary to culture clinical samples to obtain enough material. Further, the molecular techniques can be used in most laboratories without the need for specialist expertise or reagents.

The molecular typing methods of the invention may also assist in comprehensive strain identification that will be useful for epidemiological and other related studies that will be needed to monitor GBS isolates before and after introduction of GBS conjugate vaccines.

The present invention will now be described in more detail with reference to the following examples, which are illustrative only and non-limiting. The examples refer to Figures:

### DETAILED DESCRIPTION OF THE FIGURES

FIG. 1. Molecular serotype identification based on the sequence heterogeneity of the 3′-end of cpsD-cpsE-cpsF-and the 5′-end of cpsG (relevant primers are shown).

FIG. 2. Algorithm for GBS molecular serotype (MS) identification by PCR and sequencing.

FIG. 3. Multiple sequence alignments of the gene sequences of cpsG-cpsH-cpsI/M for serotypes Ia, Ib, II, III, IV, V and VI (start and stop codons are highlighted in bold).

FIG. 4. Two sites (*) of sequence heterogeneity between alp2 (AF208158, upper lines) and alp3 (AF291065, lower lines) used to distinguish them (relevant primers are shown).

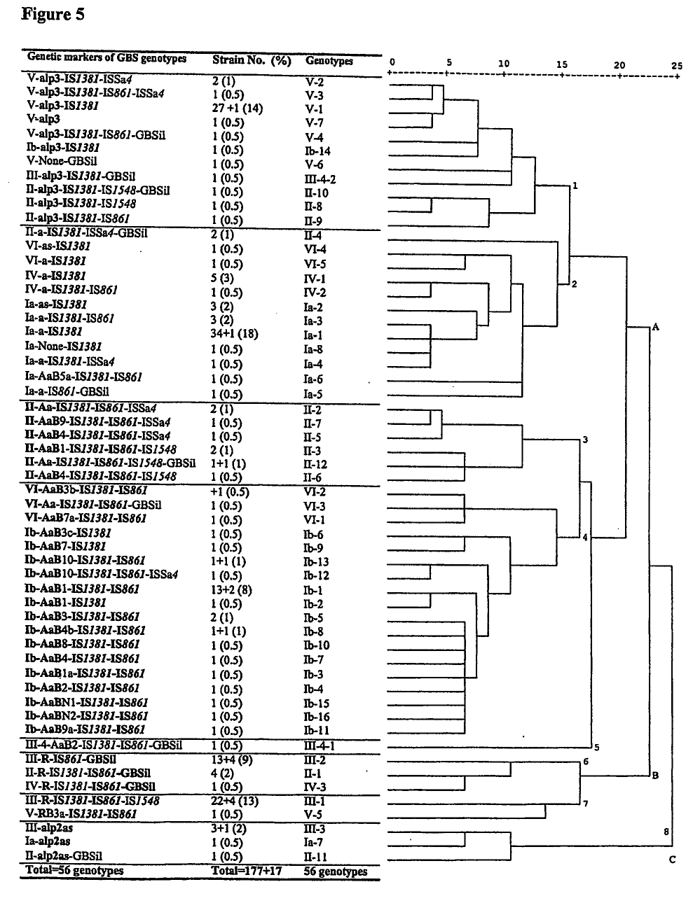

FIG. 5. Genetic relationship of 194 invasive Australasia GBS strains (or 56 genotypes).

Notes for Column Headed “Genetic Markers of GBS Genotypes”:

Protein antigen gene profile codes are:

“A”: 5′end of bca positive;

“a” or “as”: bca repetitive unit or bca repetitive unit-like region positive, with multiple or single band amplicons, respectively;

“B”: bac positive;

“R”: rib positive;

“alp2”: alp2 positive;

“alp3”: alp3 positive;

“None”: isolate contains none of the above protein genes.

The molecular markers in bold type show the common features in each cluster.

Notes for Column Headed “No. of Strains”.

After “+” are the numbers of CSF isolates, the others are blood isolates.

Notes for Column Headed “Genotypes”:

Each genotype was characterized by a distinct combination of the cps genes, protein gene profiles and mobile genetic elements. The predominant genotype in each serotype were named as the number “1” genotype of that serotype.

Notes for the Dendrogram:

At about distance 16, the 56 genotypes could be separated into 8 clusters (1-8); at about distance 22.5 the 56 genotypes could be separated into 3 cluster groups (A, B, C).

### EXAMPLES

Materials and Methods

GBS Reference Strains and Clinical Isolates.

A panel of nine GBS serotypes (Ia to VIII) was kindly provided by Dr Lawrence Paoletti, Channing Laboratory, Boston USA (reference panel 1). Dr Diana Martin, Streptococcus Reference Laboratory, at ESR, Wellington, New Zealand, provided another panel of nine international reference GBS type-strains including serotypes Ia to VI (reference panel 2) (Table 1). In addition, we tested isolates from 205 clinical cases including 146 which had been referred from various laboratories in New Zealand for serotyping and 59 isolated from normally sterile sites over a period of 10 years in one diagnostic laboratory in Sydney. One culture was subsequently shown to be mixed, so 206 different isolates were examined. Conventional serotyping (CS) was performed at the Streptococcus Reference Laboratory, at ESR, Wellington, New Zealand, and MS at the Centre for Infectious Diseases and Microbiology Laboratory Services, ICPMR, Sydney, Australia.

The two panels of GBS reference strains and 63 selected clinical isolates were studied in more detail, by sequencing >2200 base pairs (bp) of each to identify appropriate sequences for use in MS. These and the remaining clinical isolates were then used to evaluate the MS method and compare results with those of CS. Typing by both methods was done initially without knowledge of results of the other.

Bacterial isolates were retrieved from storage by subculture on blood agar plates (Columbia II agar base supplemented with 5% horse blood) and incubated overnight at 37° C.

Invasive GBS Clinical Isolates

All 194 isolates used in the study of mobile genetic elements were recovered from the blood (177) or CSF (17) of 191 patients (107 female, 80 male, four sex unrecorded; three cultures each contained mixed growth of two GBS serotypes). 108 isolates were from specimens submitted for culture to the Centre for Infectious Diseases and Microbiology Laboratory Services, ICPMR, Sydney, Australia during 1996-2001 and 83 were referred to Institute of Environmental Science and Research (ESR), Porirua, Wellington, New Zealand for serotyping, from various diagnostic laboratories in New Zealand, during 1994-2000.

Patients were classified into age-groups for analysis of genotype distribution as follows: neonatal, early onset (0-6 days); neonatal, late onset (7 days to 3 months); infant and child (4 months-14 years); young adult (15-45 years); middle-aged (46-60 years); elderly (>60 years).

These isolates are mainly a subset of the isolates described above but with reference strains and non-invasive isolates excluded.

Conventional Serotyping (CS).

CS was performed using standard methodology (Wilkinson and Moody, 1969). Briefly, an acid-heated (56° C.) extract was prepared for each isolate and the serotype determined by immuno-precipitation of type-specific antiserum in agarose. An isolate was considered positive for a particular serotype when the precipitation occurring formed a line of identity with that of the control strain. Antisera used were prepared at ESR in rabbits against serotypes Ia, Ib, Ic, II, III, IV, V and the R protein antigen. Fourteen selected isolates, including six that were nontypable using antisera against serotypes I-V, six that initially gave discrepant results between CS and MS and two separate isolates from a mixed culture, were kindly tested using antisera against all serotypes by Abbie Weisner and Dr Androulla Efstratiou at Central Public Health Laboratory, Colindale, London, UK.

Molecular Serotype Identification (MS); Development of Method.

Oligonucleotide Primers.

The oligonucleotide primers used in this study, their target sites and melting temperatures are shown in Tables 2, 6 and 10. Their specificities and expected lengths of amplicons are shown in Tables 3, 7 and 11. The primers were synthesised according to our specifications by Sigma-Aldrich (Castle Hill NSW, Australia). Four previously published oligonucleotide primers, and a series of new primers designed by us were used to sequence the genes of interest, namely 16S/23S rRNA intergenic spacer region and partial cps gene cluster, or to amplify unique sequences of individual GBS serotypes. Six previously published oligonucleotide primers and a series of new primers designed by us were used to sequence parts of and/or to specifically amplify genes encoding GBS surface proteins. We also designed a series of primers to sequence parts of and/or to specifically amplify five known GBS mobile genetic elements. Some were designed with high melting temperatures (>70° C.) to be used in rapid cycle PCR.

DNA Preparation and Polymerase Chain Reaction (PCR).

Five individual GBS colonies or a sweep of culture were sampled using a disposable loop and resuspended in 1 ml of digestion buffer (10 mM Tris-HCl, pH 8.0, 0.45% Triton X-100 and 0.45% Tween 20) in 2 ml Eppendorf tubes. The tubes containing GBS suspension were heated at 100° C. (dry block heater or water bath) for 10 minutes then quenched on ice and centrifuged for 2 minutes at 14,000 rpm to pellet the cell debris. 5 μL of each supernatant containing extracted DNA was used as template for PCR (Mawn et al., 1993).

PCR systems (25 μL for detection only, 50 μL for detection and sequencing) were used as previously described (Kong et al., 1999). The denaturation, annealing and elongation temperatures and times used were 96° C. for 1 second, 55-72° C. (according to the primer Tm values or as previously described) for 1 second and 74° C. for 1 to 30 seconds (according to the length of amplicons), respectively, for 35 cycles.

10 μL of PCR products were analysed by electrophoresis on 1.5% agarose gels, which were stained with 0.5 μg ethidium bromide mL⁻¹. For detection and/or serotype identification, the presence of PCR amplicons of expected length, shown by ultraviolet transillumination, were accepted as positive. For sequencing, 40 μL volumes of PCR products were further purified by polyethylene glycol precipitation method (Ahmet et al., 1999).

Sequencing.

The PCR products were sequenced using Applied Biosystems (ABI) Taq DyeDeoxy terminator cycle-sequencing kits according to standard protocols. The corresponding amplification primers or inner primers were used as the sequencing primers.

Multiple Sequence Alignments and Sequence Comparison.

Multiple sequence alignments were performed with Pileup and Pretty programs in Multiple Sequence Analysis program group. Sequences were compared using Bestfit program in Comparison program group. All programs are provided in WebANGIS, ANGIS (Australian National Genomic Information Service), 3^{rd} version.

Surface Protein Gene Profile Codes

Each isolate was given a protein gene profile code according to positive PCR results using various primer pairs, as shown in Table 7.

Nucleotide Sequence Accession Numbers.

The new sequence data described have been submitted to the GenBank Nucleotide Sequence Databases and allocated the following accession numbers: AF291411-AF291419 (16S/23S rRNA intergenic spacer regions for serotypes Ia to VIII reference strains from reference panel 1); AF332893-AF332917, AF363032-AF363060, AF367973, AF381030 and AF381031 (partial cps gene clusters for two panels of reference strains (Table) and selected representative clinical isolates); AF367974 (partial bac gene sequence, with an insertion sequence IS1381 from one isolate), AF362685AF362704 (partial bac gene sequences for all bac-positive isolates) and AF373214 (partial rib-like gene for reference strain Prague 25/60, an R protein standard strain).

Previously reported sequence data referred to herein have appeared in the GenBank Nucleotide Sequence Databases with the following accession numbers: AB023574 (16S rRNA gene); U39765, L31412 (16S/23S rRNA intergenic spacer regions); X68427 (S. oralis 23S rRNA gene); X72754 (cFb gene); AB028896 (cps gene cluster for serotype Ia); AB050723 (partial cps gene cluster for serotype Ib); AF163833 (cps gene cluster for serotype III); AF355776 (cps gene cluster for serotype IV); AF349539 (cps gene cluster for serotype V); AF337958 (cps gene cluster for serotype VI); M97256 (bca gene); X58470, X59771 (bac gene); U58333 (rib gene); AF208158 (alp2 gene), AF291065-AF291072 (alp3 gene); AF064785 (IS1381); M22449 (IS861); Y14270 (IS1548); AF064785 (IS1381); AF165983 (ISSa4); and AJ292930 (GBSi1).

Statistical Analysis and Dendrogram.

SSPS version 11 software was used for statistic analysis. A dendrogram was formed using Average Linkage (between groups) and Hierarchical Cluster Analysis in SSPS version 11 software. The presence or absence of each marker—MS Ia, Ib, II, IV-VI , sst III-1-4; pgp “A”, “R”, “a”, “as”, “alp2”, alp3”; bac subgroups 1, 1a, 2, 3, 3a, 3b, 3c, 4, 4b, 5a, 7, 7a, 8, 9, 9a, 10, n1, n2; and mge IS 1381, IS861, IS 1548, ISSa4, GBSiI—were Included in the analysis. The genotypes were each characterized by a distinct combination of the molecular serotyping (MS) or sst, pgp and mge.

### Example 1

### Study of inter- and intra-serotype/serosubtype Sequence Heterogeneity in Specific Regions of the GBS Genome and Assessment of Suitability for Molecular Serotyping/Serosubtyping

Polymerase Chain Reaction.

With two exceptions, all GBS-specific primer pairs produced amplicons of the expected size from all reference strains and clinical Isolates tested (Table 3). The exceptions were Sag59/Sag190 and CFBS/CFBA Both target the cfb gene, but failed to produce amplicons from one clinical Isolate, despite repeated attempts. We assumed that this isolate either lacked the cfb gene or that the gene was present in a mutant form. It has been suggested previously that PCR targeting the cfb gene will not identify all GBS isolates (Hassan et al., 2000) and that another primer pair based on 16S rRNA gene, DSF2/DSR1 (Ahmet et al., 1999) was not entirely specific. Therefore, in this study, we used both primer pairs (DSF2/DSR1 and Sag59/Sag190) to confirm all the isolates were GBS.

Sequence Heterogeneity of 16S/23S rRNA Intergenic Spacer Regions,

The 16S/23S rRNA intergenic spacer regions were sequenced for the serotypes Ia to VIII from reference panel 1. Multiple sequence alignment showed differences between serotypes at only two positions: 207 (serotype V is T or C [T/C], serotypes VII and VIII are C, others are T) and 272 (serotype III is T, others G). These regions are therefore unsuitable for MS.

Sequence Heterogeneity at the 3′-end of cpsD-cpsE-cpsF-and the 5′-end of cpsG.

Using a series of primers targeting the 3′-end of cpsD-psE-cpsF-and the 5′-end of cpsG, we amplified and sequenced 2226 or 2217 bp—depending on the presence or absence of a nine-base repetitive sequence—from both panels of reference strains (serotypes Ia to VII) and 63 selected clinical isolates. Representative sequences were deposited into GenBank. See Table 1 for GenBank accession numbers of reference panel strains.

Repetitive Sequence.

At the 3′-end region of cpsD, we found a nine-base repetitive sequence (TTA CGG CGA) in most isolates of MS Ia and II, some of MS III, all of MS IV, V, and VII, but none of the isolates of MS Ib or VI examined. (Table 4). The presence or absence of this repetitive sequence can be used to further subtype MS Ia, II and III (see below).

Intra-Serotype Heterogeneity.

In general, intra-serotype heterogeneity was low—there were minor random variations in a few isolates of all serotypes except MS III, in which the intra-serotype heterogeneity was more complex. MS III could be divided into four sequence subtypes on the basis of heterogeneity at 22 positions—62, 139, 144, 204, 300, 321, 429, 437, 457, 486, 602, 636, 971, 1026, 1194, 1413, 1501, 1512,1518, 1527, 1629, and 2134—and the presence or absence of the repetitive sequence (at 78-86) (Table 4).

Among 60 MS III isolates (58 clinical isolates and two reference strains), serosubtypes III-1 (30 isolates) and III-2 (22 isolates) were predominant. The repetitive sequence was present in serosubtype III-1 but not III-2; there were differences at seven other sites (139, 144, 204, 300, 321, 636, and 1629).

There were five isolates belonging to serosubtype III-3, which contained the repetitive sequence and were identical with serosubtype III-1 at three variable sites (139, 144, and 300) and with serosubtype III-2 at four (204,321, 626 and 1629). Seroubtype III-3 differed from both serosubtypes III-1 and III-2 at seven sites (486, 1026, 1413, 1512, 1518, 1527, and 2134). These seven sites in serosubtype III-3 were identical with the corresponding sites of MS Ia.

There were three serosubtype III-4 isolates, whose sequences were nearly identical with the corresponding sequence of MS II. The only exception was at position 437, where the nucleotide was T in serosubtype III-4 (as in MS VII), and C in MS II. This difference can be used (in addition to PCR, see below) to differentiate serosubtype III-4 from MS II. Two serosubtype III-4 isolates contained the repetitive sequence, and the other did not. Because of the small number of serosubtype III-4 isolates, we did not use the repetitive sequence to subtype them further.

Inter-Serotype Heterogeneity.

There were 56 sites of heterogeneity between the eight MS (Table 4). The most suitable sites, for use in PCR/sequencing for MS, were a group of 23 sites nearest to the 3′-end of the region (Table 4, FIG. 1). Firstly, they were consistent across two panels of reference strains and most clinical isolates (the only exceptions were the small number of serosubtypes III-3 and III-4 isolates, see below). Secondly, they were relatively concentrated within a 790 bp region, which is a convenient length for sequencing in a single reaction. Thirdly, they contained enough heterogeneity sites to allow differentiation, with few exceptions, of MS Ia-VII. Based only on this 790 bp region, serosubtype III-3 cannot be distinguished from MS Ia, nor serosubtype III-4 from MS II. However, they can be identified by MS III-specific PCR (see below).

Serotype VIII does not form amplicons with primer pairs targeting the 790 bp region, but can be identified by exclusion after PCR identification of GBS. In this study, one MS VIII isolate was identified, for which none of the primer pairs that amplify the 2226 bp region (in addition to those that amplify the 790 bp region) produced amplicons. This result was confirmed by the use of serotype VIII-specific antiserum.

Mixed Serotype-Specificities in Single Isolates.

Eleven isolates were identified as one MS on the basis of the MS-specific PCR and overall sequence (within the 2226/2217 bp segment) but their sequences differed at some sites from isolates of the same MS and shared site-specific characteristics of another. They included five serosubtype III-3 isolates and three serosubtype III-4 (see above). One non-serotypable reference strain (Prague 25/60), which was identified as MS II, differed from other MS II isolates at five sites at the 5′-end of the region, and was identical with MS III at three of these sites. Prague 25/60 MS III-specific PCR was negative. One clinical isolate identified as CS II, and MS II on the basis of its overall sequence, had bases at nine sites at the 5′-end of the region, that were characteristic of serotype Ib; MS Ib-specific PCR was negative. Finally, one CS V reference strain (Prague 10/84) had the same sequencing result as the corresponding sequence in GenBank (AF349539), but both were different, at three sites at the 5′-end of the region, from sequences of the other MS V strains that we studied.

All of these mixed-serotype specificities, except for those associated with serosubtypes III-3 and III-4, occurred at the 5′-end region of the 2226/2217 fragment. This supported our selection of the 790 bp 3′-end as the sequencing target for MS. Using this target, all MS were correctly identified except for MS III belonging to serosubtypes III-3 and III-4, which can be identified by MS III-specific PCR (see Example 2).

### Example 2

### Molecular Serotype Identification (MS) Based on MS-Specific PCR Targeting the 3′-end of cpsG-cpsH-cps I/cpsM

Our sequence alignment results showed that there was significant sequence heterogeneity in the 3′-end of cpsG-cpsH-cps I/cpsM (FIG. 3), which makes it appropriate for use in the design of specific primer pairs for differentiation of serotypes Ia, Ib, III, IV, V, and VI directly by PCR. To fulfil possible additional future requirements—for example, development of multiplex PCR and/or to allow further evaluation of the sequence typing method, we designed several primer pairs for each serotype (Tables 2 & 3). Using two panels of reference strains and the specified conditions, all primer pairs amplified DNA only from the corresponding serotypes. When clinical isolates were tested, similar results were obtained with two sets of MS-specific primer pairs. In general, more stringent conditions (lower primer concentration, higher annealing temperatures) could be used with primers generating smaller amplicons. Those selected for MS are shown in Table 3 and FIG. 2.

A MS was assigned, by PCR, to 179 of 206 (86.9%) clinical isolates as follows: MS Ia 40; MS Ib 35; MS III 58 (including those previously identified as serosubtypes III-3 and III-4); MS IV 7; MS V 36; MS VI 3.

### Example 3

### Comparison of Serotype Identification Results between MS and CS

After CS and MS had been completed, the results were compared. Initial results were discrepant for 15 isolates, all but five of which (see below) were resolved by retesting and/or correction of clerical errors.

The CS and MS/sequence subtyping results are shown in Table 5. A MS was assigned to all isolates by PCR and/or sequencing, compared with 188 of 206 (91.3%) by CS. Specific PCR has not yet been developed for MS II and VIII, so all MS II isolates were determined by sequencing only and one presumptive MS VIII isolate was decided by exclusion (see Example 1). For all other isolates, the results of PCR and sequencing were consistent, except for serosubtypes III-3 and III-4 and other minor sequence differences described above (Example 1). CS results correlated well with PCR results.

Final CS and MS results were the same for all 188 isolates (100%) for which results for both methods were available. Eighteen clinical isolates that were non-serotypable by CS, were assigned MS as follows: Ia, two; Ib, five; II, one; serosubtype III-1, three; serosubtype III-2, one; V, five; and VI, one.

Sequences (2217 bp) of three clinical isolates that we identified as MS VI, were identical with those for serotype VI reference strains and the corresponding sequence in GenBank (AF337958).

Mixed Culture.

Four clinical isolates gave positive results with MS III-specific PCR, but were provisionally identified as MS II by sequencing. Three were CS III and one CS II, with a weak cross-reaction with serotype III antiserum. These isolates were studied further by subculturing 12 individual colonies of each. All subcultures were tested by MS III-specific PCR. All 12 colony subcultures of the three CS III isolates were positive by MS III-specific PCR and the isolates were therefore classified as serosubtype III-4 (see above). However, 11 of 12 colony subcultures of the fourth isolate were negative by MS III-specific PCR; and one was positive by MS III-specific PCR. It was therefore assumed that this was a mixed culture, predominantly of MS/CS II. The one MS III-specific PCR positive colony was subsequently identified as serosubtype III-2 and included as an additional clinical isolate (total 206 in all).

### Example 4

### Algorithm for Serotype Assignment of GBS by PCR and Sequencing

As an example of how the PCR and sequencing methods described above may be used clinically to perform GBS serotype identification, we designed an algorithm for clinical use. All the primers (except the inner sequencing primers) used were given high melting temperature (>70° C.), so rapid cycle PCR could be used (FIG. 2) (see Table 2 for primer sequences).

### Example 5

### Identification of Regions in the alp2, alp3 and Rib Genes Suitable for Protein Antigen Gene Specific Subtyping

Polymerase Chain Reactions.

With few exceptions, all primer pairs produced amplicons of predicted length from isolates giving positive results (Table 7). The exceptions included one isolate that was positive by PCR using primer pairs GBS1360S/GBS1937A and GBS1717S/GBS1937A (which both target bac gene) but produced amplicons significantly longer than those of other bac gene-positive isolates. Sequencing showed that the amplicon contained the insertion sequence IS1381 with minor variations compared with the published sequences (Tamura et al., 2000). The amplicons produced using primers IgAagGBS/RlgAagGBS and IgAS1/IgAA1 (also targeting bac gene) varied in length (Berner et al., 1999) and were sequenced for further subtyping (see below and Table 8).

Amplicon Sequencing Results.

To confirm the specificity of selected primer pairs that we had designed or modified, we sequenced 10 of 23 amplicons produced by bcaS1/bcaA (targeting the 5′-end of bca gene) and all of those produced by ribS1/ribA3 (targeting rib gene) and GBS1360S/GBS1937A (targeting bac gene), from the two panels of reference strains and 31 randomly selected clinical isolates.

All 10 amplicons of primers bcaS1/bcaA and 12 of 13 of primers ribS1/ribA3 were identical with the corresponding gene sequences in GenBank (M97256, bca gene and U58333, rib gene, respectively). One additional isolate, namely Prague 25/60 in reference panel 2 (which is used to raise R antiserum), produced an amplicon with primer pair ribS1/ribA3 only at a lower annealing temperature (55° C.) but not with ribS2/ribA1 and ribS2/ribA2. It was therefore assumed not to contain rib gene, although the amplicon sequence showed considerable homology with rib gene (71.4% or 66.6% according to whether or not the primer sequences were included) (FIG. 3). This isolate was the only one, of 224 tested, for which PCRs were negative using ribS2/ribA1 and ribS2/ribA2 but positive using ribS1/ribA3. The latter primer pair is assumed to be not entirely specific for rib gene and was therefore used only for sequencing.

Four of 10 amplicons of primer pair GBS1360S/GBS1937A (targeting bac gene) were identical with the corresponding sequence in GenBank (X58470, X59771). A single point mutation (A to G, 1441 of X59771) was found in the remaining six bac gene amplicons, including the one which contained the insertion sequence IS1381 (see above and AF367974).

Amplicons from all of the 224 isolates that gave positive PCR results using primer pairs bcaS1/balA (targeting alp2/alp3 genes), bal23S1/bal2A2 (targeting alp2 gene) and IgAagGBS/RlgAagGBS (targeting bac gene) were sequenced.

Fifty isolates produced amplicons using primer pair bcaS1/balA. The sequences of nine were identical with the corresponding portions of the published sequence of alp2 gene (AF208158) and 41 with that of alp3 gene (AF291065). There are two consistent heterogeneity sites between alp2 and alp3 genes in the sequences of bcaS1/balA amplicons (FIG. 4), which can be used to distinguish them, in addition to alp2 and alp3 gene-specific PCR. All nine amplicons of primer pair bal23S1/bal2A2 were identical with the corresponding portion of the alp2 gene sequence in GenBank (AF208158).

The primer pair IgAagGBS/RIgAagGBS identified bac gene in 52 isolates. There was considerable sequence variation, which allowed separation of bac gene-positive isolates into 11 groups and 20 subgroups based on amplicon length and sequence heterogeneity, respectively (Table 8). The groups contained small numbers (one to five) of isolates except for B1 (20 isolates, 2 subgroups) and B4 (11 isolates, 3 subgroups). The differences in amplicon length was generally caused by the presence or absence of short repetitive sequences.

Further Confirmation of Specificity of Surface Protein Gene-Specific Primer Pairs.

To confirm primer specificity, we compared the results of PCR using the primer sequences we had designed or modified for bac gene PCR, with those of PCR using previously published primers and found 100% correlation.

The previously reported non-specificity of the published primer pair bcaRUS/bcaRUA (targeting the bca gene repetitive unit) was confirmed. Using these primers, all nine alp2 gene positive (bcaS1/bcaA negative) isolates and 53 which were PCR negative using the primers bcaS1/bcaA, bcaS2/bcaA (targeting the 5′-end of bca gene), bal23S1/bal2A2 and bal23S2/bal2A1 (targeting the 5′-end of alp2 gene) produced amplicons. Our sequencing showed that bca gene and alp2 gene have significant homology in the regions targeted by bcaRUS/bcaRUA allowing amplicon formation from alp2 gene-positive strains. These false positive results could be due to the presence of other C alpha-like proteins, containing regions homologous with the bca gene repetitive unit (bca gene repetitive unit-like sequence).

We also showed that the results of PCR using two or more primer pairs that we had designed for individual genes (rib, alp2, and alp3 genes) correlated well, supporting the specificity of each set. The only exception, as mentioned above, was ribS1/ribA3, which produced a non-specific amplicon from one of 224 isolates tested.

### Example 6

### The Relationship Between Surface Protein Antigen Gene Profiles and cps Serotypes/Serosubtypes

Surface Protein Gene Profiles.

For each gene (except bca gene repetitive unit or bca gene repetitive unit-like region), we selected two primer pairs to identify and characterise GBS surface protein by PCR. Each isolate was given a protein gene profile code according to PCR results as follows:

“A”: 5′end of bca gene amplified by bcaS1/bcaA and bcaS2/bcaA;

“a” or “as”: bca gene repetitive unit or bca gene repetitive unit-like region amplified by bcaRUS/bcaRUA, with multiple or single band amplicons, respectively;

“B”: bac gene amplified by GBS1360S/GBS1937A and IgAagGBS/RlgAagGBS (>20 subgroups based on sequence heterogeneity).

“R”: rib gene amplified by ribS2/ribA1 and dbS2/ribA2;

“alp2”: alp2 gene amplified by bal23S1/bal2A2 and bal23S2/bal2A1 and

“alp3”: alp3 gene amplified by bal23S1/bal3A and bal23S2/bal3A (Table 7).

Four common profiles accounted for 203 of 224 (90.6%) isolates: “R” (62 isolates), “AaB” (51 isolates), “a” (49 isolates) and “alp3” (41 isolates) (see Table 4). Only two isolates contained no surface protein gene markers. All but one isolate with the bac gene (“B”) also had bca gene, with its repetitive unit (“Aa”); one had rib gene. All “alp2” isolates contained single bca repetitive unit-like sequences (“as”). “A”, “R”, “alp2” and “alp3” were all mutually exclusive. 62 of 63 isolates with rib gene (“R”) and 41 of 41 isolates with alp3 gene had no other protein antigen markers.

The Relationship Between Surface Protein Antigen Gene Profiles and cps Serotypes/Serosubtypes.

A cps molecular serotype (MS) was assigned to all isolates in accordance with the methods described in Examples 1 to 4 and the results correlated with conventional serotyping (CS) results except for 19 of 224 isolates that were nontypable using antisera. The relationship between surface protein gene profiles and cps MS are summarised in Table 9.

The following strong associations were confirmed or demonstrated between: MS Ia and bca gene repetitive unit or bca gene repetitive unit-like sequence (most with profile “a”); MS serosubtypes III-1 and III-2 and rib gene; MS serosubtype III-3 and alp2 gene; MS Ib and bca/bac genes and MS V and alp3 gene. MS II showed the most varied surface protein gene profiles. However, the relationships were not absolute and different combinations of cps serotypes and protein gene profiles produced 31 different serovariants or 51 when bac gene (“B”) subgroups were considered.

### Example 7

### The Relationship Between Surface Protein Antigens and Protein Gene Profiles

Based on conventional serotyping, 33 isolates (belonging to CS Ia/c, Ib/c, IIc, IIb, IIIc or IIIb) reacted with the C antiserum. The surface protein gene profiles of all these isolates contained bca gene (“A”) or bca gene repetitive unit-related markers (“a” or “as”): Aa, 3; AaB, 18; a, 11; alp2as,1. Twenty nine isolates reacted with the R antiserum and, of these, 22 contained rib gene and six, alp3 gene. The strain used to raise the R protein antiserum (Prague 25/60) contained a presumed rib-like gene (see above and FIG. 3).

### Example 8

### Identification of Mobile Genetic Elements Suitable for Molecular Subtyping

We developed a series of PCR primers to screen for the presence of five mobile elements in GBS serotypes.

Specificity of Primers Pairs.

All the primer pairs produced amplicons of the expected lengths (Table 11) from some reference and/or some clinical isolates (Table 12). To evaluate the specificity of our primer pairs, we sequenced all amplicons produced by primers IS1548S/IS1548A3 and ISSa4S/ISSa4A2, and amplicons, selected from both reference and clinical isolates, produced by IS861S/IS861A2 (12 isolates), IS1381S1/IS1381A (24 isolates) and GBSi1S1/GBSi1A2 (11 isolates).

All 41 IS1548 and 15 ISSa4 amplicon sequences were identical with the corresponding sequences in GenBank (Y14270 and AF165983, respectively). Five of 12 IS861 amplicon sequences were identical with the corresponding IS861 sequence in GenBank (M22449). The other seven differed, at position 732, from the published sequence (G to A) and the reference strain Prague 25/60 had two additional differences—G to A and T to A—at positions 576 and 830 of M22449, respectively.

Previously, we found a full-length insertion sequence IS1381 (AF367974) within C beta antigen gene of a clinical isolate, with several differences compared with the original published sequence (AF064785): the terminal inverted repeats contained 15, rather than 20 base pairs (bp); there was a three bp deletion and four individual bp differences in the putative transposase pseudogene between positions 419 to 429 (of the original GenBank sequence)—GGG ATC CGA TT (AF064785) vs CAG A- -GG TA (AF367974; our sequence). All amplicons of primer pair IS1381S1/IS1381Afrom 12 reference and 12 selected clinical isolates were identical with each other and with that of our IS1381 sequence in GenBank (AF367974) but different, as above, from the original reported IS1381 sequence (AF064785).

The amplicons of primer pair GBSi1S1/GBSi1A2 from all four GBSi1-positive reference strains and seven selected clinical isolates were sequenced. Six (including those of three reference strains) were identical with the corresponding GBSi1 sequence in GenBank (AJ292930). Amplicons from four clinical isolates showed three site-variations (C to T at position 767, A to C at position 846 and T to C at position 923 of AJ292930 sequence). The reference strain Prague 25/60 showed only the first two of these site-variations.

In addition to sequencing, we evaluated the specificity of our primer pairs by comparing PCR results for two or more primer pairs for each target (Table 11). In all cases, the same sets of isolates gave positive results when tested with PCR targeting the same mobile genetic elements, thus confirming the specificity of the primer pairs.

PCR Results Using Specific Primer Pairs for All Five Mobile Genetic Elements.

IS861, IS1548, IS1381, ISSa4 and GBSi1 were identified in 55%, 18%, 85%, 7% and 19% of isolates, respectively. None of the mobile elements was detected in 10 (4%) isolates. The distributions of the five mobile elements identified by PCR in the 224 GBS isolates tested in the previous examples are shown in Table 12. IS1381 was detected alone in 79 isolates and GBSi1 alone in one. Forty-six isolates contained two different insertion sequences (IS861 and IS1381, 42 isolates; IS1548 and IS1381, three isolates; ISSa4 and IS1381, one isolate). Forty-four isolates contained three (IS861, IS1548 and IS1381 34; IS861, ISSa4 and IS1381, 10) and one contained all four insertion sequences. Forty-one isolates contained GBSi1 in combination with one (IS861, 22; IS1381, one isolate) two (IS861 and IS1381, 11; ISSa4 and IS1381, three isolates) or three (IS861, IS1548 and IS1381, four isolates) insertion sequences.

PCR Results for the 194 Invasive Isolates Using Specific Primer Pairs for All Five Mobile Genetic Elements.

The numbers of isolates containing different mobile genetic elements (mge) combinations (from none to four per isolate) are shown in Table 13. IS1381, IS861, IS1548, ISSa4 and GBSi1 were identified in 87%, 52%, 17%, 6% and 18% of isolates, respectively. Six (3%) isolates contained no mge.

### Example 9

### The Relationships Between cps Serotypes, Serosubtypes, Surface Protein Gene Profiles and Mobile Genetic Elements

The distribution of each of the five mobile genetic elements in different cps serotypes, serotype III subtypes and surface protein gene profiles are shown in Tables 12 and 13. The most consistent findings for each sero/serosubtype were:

1) Serotype Ia—most (>80%) expressed proteins that closely related with C alpha protein and contained IS1381

2) Serotype Ib—most (>90%) expressed C alpha and C beta proteins and contained IS861 and IS1381

3) Serotype II—exhibited two common patterns:

a) >50% expressed C alpha protein (and often C beta) and contained IS861, IS1381 and sometimes other mobile elements, especially ISSa4 or

b) >25% expressed Rib protein and contained IS861, IS1381 and GBSi1

4) Serosubtype III-1—all expressed Rib protein and contained IS861, IS1548 and IS1381 but not GBSi1.

5) Serosubtype III-2—all expressed Rib protein and contained IS861 and GBSi1 but neither IS1548 nor IS1381.

6) Serosubtype III-3—all expressed C alpha-like protein 2 and contained no mobile genetic elements.

7) Serosubtype III-4—expressed various proteins; all contained GBSi1.

8) Serotype IV—most expressed proteins that closely related with C alpha protein and contained IS1381

9) Serotype V—most expressed C alpha-like protein 3 contained IS1381

10) GBSi1 and IS1548 were mutually exclusive in serotype III (III-1, III-2 and III-4) but not in serotype II.

11) All isolates that expressed C alpha-like protein 2 contained no insertion sequences.

Predominant Relationships between MS/sst, pgp and mge.

FIG. 5 shows the relationships between the various genetic markers. IS1381 was present in nearly all isolates of MS Ia, Ib, IV, V and VI, but in none of sst III-2 or III-3. IS1548 was found exclusively, and GBSil most commonly, in serotypes II or III; three isolates (all MS II) contained both GBSi1 and IS1548. IS861 was found in all sst III-1 and III-2 and most MS II and Ib isolates but only in 14% of other MS isolates. ISSa4 was present in only 6% of isolates, more than half of which were MS II; it was present in one invasive isolate obtained before 1996 (1994). IS1381 was found in most isolates except those in cluster 8, pgp “alp2”, which had no insertion sequences. IS861was found in most genotypes with pgp “AaB” (clusters 3 and 4) and all genotypes with pgp “R” (clusters 6 and 7).

Genotypes Based on MS/sst, pgp, bac Subtypes and mge.

MS/sst, pgp, bac subtype (for isolates with pgp “B”) and the presence of various combinations of mge provide a PCR/sequencing-based genotyping system. The 194 invasive isolates in this study represented seven serotypes, ten MS/sst, 41 subtypes based on the distributions of pgp and mge or 56 genotypes when bac subtypes (mainly in MS Ib) were included (FIG. 5).

Theoretical GBS Clonal Population Structure.

Theoretically there are 13 possible GBS MS/sst (eight MS—Ia, Ib, II, IV-VIII, four sst III 1-4 and cps gene cluster absent) and at least 10 pgp (none, “Aa”, “AaB”, “a”, “as”, “R”, “RB”, “alp2as”, “alp3” or “alp4a”). If the 22 bac subgroups identified so far are included, there are up to 31 pgp. If the five mge were independently, randomly distributed and present or absent, there would be 13×31×2⁵=12,896 different possible combinations of molecular markers. The fact that only 56 different combinations were found (FIG. 5), demonstrates that markers are not randomly distributed or, in other words, these invasive Australasian GBS isolates have a clonal population structure. It is possible, but unlikely, that these isolates represent a very limited number of GBS genotypes.

The Phylogenetic Relationship of Australasian Invasive GBS.

The 56 genotypes formed eight clusters, separated at a genetic distance of about ˜16 (or three cluster groups separated at a distance of ˜22.5). The pgp was the main determinant of cluster separation (FIG. 5). 94% of isolates belonged to five MS (Ia, Ib, II, III and V), 62% belonged to five (9%) genotypes (Ia-1, Ib-1, III-1, III-2, V-1) and 92% belonged to the five largest clusters (1, 2, 4, 6 and 7). Cluster group A, the largest, contained 139 (72%) isolates and 48 (86%) genotypes, 45 of which contained fewer than five isolates, whereas cluster group B contained 49 (25%) isolates and five (9%) genotypes.

The main characteristics of each cluster were as follows:

Cluster 1. “alp3”, IS1381 (39 isolates, four MS, 11 genotypes; predominant genotype V-1).

Cluster 2: “a” or “as”, IS1381 (55 isolates, four MS, 12 genotypes, predominant genotype Ia-1).

Cluster 3: “Aa” or “AaB”, MS II, IS1381, IS 861 (10 isolates, six genotypes).

Cluster 4: “AaB”, IS1381, IS861 (35 isolates, two MS: VI or Ib; 18 genotypes; predominant genotype Ib-1).

Cluster 5. “AaB”, IS861, GBSi1, genotype III-4-1 (one isolate).

Cluster 6: “R”, IS861 and GBSil (22 isolates, three MS/genotypes; predominant genotype III-2).

Cluster 7: “R”, IS1381 and IS861 (27 isolates; two MS/genotypes; predominant genotype III-1).

Cluster 8: “alp2as”, no IS (six isolates; three MS/genotypes; one contained GBSi1).

The phylogenetic study showed that the dendrogram inferred by SSPS was very robust.

The Relationship Between Genotypes and GBS Disease Patterns.

The distribution of MS and genotypes in different age groups of patients with invasive GBS disease is shown in Table 14. All common MS were represented in more than one patient group. However, there were highly significant associations (when compared with all other age-groups) between sst III-2 and late onset neonatal infection (p=0.0005) and MS V and infection in the elderly (p=0.001).

There were 17 isolates from cerebrospinal fluid specimens, nine (53%) of which were MS III (from three different sst/genotypes, each in a different cluster). The other eight isolates were distributed among five MS, seven genotypes and four clusters. Meningitis occurred in all age-groups but comprised 23% of cases in the late onset neonatal group compared with 5% in all other groups.

Discussion

Capsule production in GBS is controlled by capsular polysaccharide synthesis (cps) gene cluster, which had been sequenced for serotype Ia and serotype III before we began our study. Corresponding sequences for serotype Ib (Miyake et al., 2001 submitted into GenBank, GenBank accession number: AB050723), and for serotypes IV, V, and VI (McKinnon et al., 2001 submitted into GenBank, GenBank accession numbers: AF355776, AF349539, AF337958, respectively) were released recently when the project was nearly finished but those for the other three serotypes (II, VII and VIII), the sequences of cps gene clusters, have not been published previously.

The sequences of cps gene clusters for serotypes Ia, and III showed considerable homology at the 3′-end of cpsD cpsE-cpsF-and the 5′-end of cpsG. We designed a series of primers to amplify a 2226/2217 bp segment in this region and found that amplicons were obtained from all serotypes except VIII. This confirmed a previous suggestion that serotype VIII is significantly different from other serotypes in this region.

Using eight serotype (Ia to VII) reference strains, we showed more than 50 heterogeneity points between serotypes (FIG. 1, Table 4). Using 63 selected clinical isolates that had been serotyped by conventional methods, we found that these inter-serotype differences were generally consistent and specific, especially the 23 sites clustered at the 3′-end of the regions. We used these differences to assign serotypes to the remaining clinical isolates collected in this study, without knowledge of the serotype obtained by conventional methods.

Sequence analysis of the 3′-end of cpsG-cpsH-cpsI/cpsM for serotypes Ia, III, Ib, IV, V and VI showed that this region is highly variable (FIG. 3), making this region a suitable target for direct serotype identification by PCR. We designed several pairs of MS-specific primers for MS Ia, Ib, III, IV, V and VI and used them to test two CS reference panels. Selected primer pairs were used for MS, by PCR alone, of 86.9% of our 206 clinical isolates. Using rapid-cycle MS-specific PCR, results are available within one working day. In future, it will be possible to extend this method to all MS, when cps gene cluster sequences in this region are available for serotypes II, VII and VIII. Meanwhile, MS II and VII can be identified by sequencing the 790 bp PCR amplicons of the 3′-end of cpsE-cpsF-the 5′-end of cpsG (FIG. 1, Table 4). A positive GBS-specific PCR and negative PCR results with all the primers that amplify the 790 bp, identified MS VIII, by exclusion.

In future, and in some laboratories currently, sequencing of the 790 bp PCR amplicons of the 3′-end of cpsE-cpsF-the 5′-end of cpsG for all isolates may be more convenient, as only one method and fewer primers are needed. However, if sequencing is not available in-house, the turn-around time is longer and a small proportion of serotypes would be wrongly assigned (serosubtypes III-3 and III-4 as MS Ia and II, respectively). This could be avoided by screening with MS III-specific PCR first. Sequencing the 790 bp PCR amplicon, allows MS III to be subtyped on the basis of the sequence heterogeneity.

Previous studies have shown that serotypes Ia, Ib, II, III, and V are those most frequently isolated from normally sterile sites, in the United States and several countries. Serotypes VI and VIII are the predominant serotypes isolated from patients in Japan, but are uncommon elsewhere. Although our isolates were selected, they were probably representative of those causing disease in Australasia; Ia, Ib, II, III, and V were the most common serotypes identified, although there were small numbers of serotypes IV,VI and, VIII.

Up to 13% of GBS isolates are non-serotypable and in our study the proportion was 8.7% (18/206) using the antisera available. This may be due to decreased type-specific-antigen synthesis; non-encapsulated phase variation; or insertion or mutation in genes of cps gene clusters. One non-serotypable strain GBS in our study had a T base deletion in cpsG gene, which caused a change in the cpsG gene reading frame.

We have also developed PCR-based methods to identify GBS surface protein genes and further characterise these isolates. Using the published bac gene sequence, we modified bac gene-specific primers and designed new primers, with high melting temperatures (>70° C.) suitable for rapid cycle PCR targeting all major surface protein genes.

As previously reported, a published PCR primer pair targeting the bca gene repetitive unit (at the 3′-end of bca gene), was not entirely specific for bca gene. We designed two new primer pairs targeting the 5′-end of bca gene, to improve the specificity. However, very few serotype Ia strains gave positive results using these primers whereas all were PCR positive using primers targeting the bca gene repetitive unit. These results were consistent with a previous report, that a probe targeting the 5′-end of bca gene hybridized with only one of nine serotype Ia strains, but a large bca gene probe, including the tandem repeat region, hybridized with all nine strains.

PCR specific for rib, alp2 and alp3 genes has not been described previously. The primer pairs we designed mainly targeted the 5′-ends of the gene and were chosen after comparing the gene heterogeneity with related gene sequences. We designed two or more primer pairs for each gene to check primer specificity by comparison of results of different PCR targeting the same genes. Protein gene profiles “alp2” and “alp3” were distinguished on the basis of the alp2 and alp3 gene-specific PCR and/or two sequence heterogeneity sites in the amplicons of bcaS1/balA, or bcaS2/balA.

To confirm the specificity of our primers, we used them to examine two reference panels and selected GBS isolates. The longest amplicons produced by PCR for each gene were sequenced, to provide maximal sequence information and ensure that the inner primers were not located at strain heterogeneity sites. Our sequencing results confirmed the specificity of the primers. Two pairs of primers for each gene were compared, with similar results. Finally, six gene/region specific primer pairs (including the one targeting the bca gene repetitive unit) were used to define protein antigen gene profiles for all 224 isolates.

The study showed that only one member of the surface protein gene family containing repetitive sequences—rib, bca, alp2, and alp3 genes-could be present in any single isolate. However, all isolates containing bac gene, which is not a member of the surface protein gene family containing repetitive sequences, also contained either bca gene (51/52) or rib gene (1/52).

Bac gene was present in 23% of isolates, a similar proportion to that (19-22%) previously reported. In common with others, we found variations in the bac gene due to variable small internal repetitive sequences. These bac gene repetitive sequences were irregular (unlike those of the bca-rib gene family). Their role is not clear, but they are potentially useful molecular markers for epidemiological studies.

Our data show that some serotype III isolates (our MS serosubtypes III-1 and III-2) were closely associated with rib gene, and others (our MS serosubtype III-3) with alp2 gene. Serotype Ib was associated with bca and bac genes and serotype V with alp3 gene. However, as the relationship was not absolute, different combinations of cps serotypes-serosubtypes/protein gene profiles identified many serovariants, which will be useful in epidemiological studies and in formulation of conjugate vaccines. Based on PCR only, we were able to divide our 224 isolates into 31 serovariants based on bac gene (B) groups or 51, based on subgroups. Theoretically, there are likely to be additional serovariants.

We found that the antisera to “c” and “R” protein antigens were not entirely specific for any particular protein genes. However, reaction with “c” antiserum mostly reflected the presence of genes encoding C alpha (bca gene) and related protein antigens (at least including alp2 gene) and the antiserum to “R” with those encoding Rib (rib gene) and related proteins (at least including alp3 gene, and the rare presumed rib-like gene).

We have also investigated the presence of a number of mobile element in different serotypes of GBS. Four different insertion sequences have been identified previously in GBS. Multiple copies of IS861 in some serotype III isolates were associated with increased capsule gene expression. We found IS861 in all serosubtypes III-1 and III-2 and most serotype II and Ib isolates but few others. All IS861-containing isolates contained at least one additional mobile element.

Multiple copies of IS1381 have been found in a high proportion GBS and other Streptococcus species, including S. pneumoniae and used as probes for restriction fragment length polymorphism (RFLP) analysis of GBS for epidemiological studies (Tamura et al., 2000). We found IS1381 in 85% of isolates overall. They were present in all isolates of serosubtype III-1 but none of serosubtypes III-2 or III-3. Our IS1381 sequences, from 24 isolates, were identical with each other, but differed at several sites, from that previously described (AF064785). The significance of these differences is unknown, but it emphasizes the importance of confirming sequences from as many different strains as possible.

ISSa4 was first identified in a nonhemolytic GBS isolate, in which it caused insertional inactivation of the gene cylB, which is part of an ABC transporter involved in production of hemolysin. Only a small proportion of (mainly hemolytic) GBS isolates (4%) contained ISSa4, all of which had been isolated since 1996 and it was postulated that ISSa4 had been newly acquired by GBS. We also found ISSa4 in only a small proportion of isolates (7%) but it was present in similar proportions of clinical isolates obtained before (4 of 44) and during or after (11 of 162)1996.

IS1548 was first discovered in some hyaluronidase-negative GBS serotype III isolates, in which it caused insertional inactivation of the gene hylB (one of a cluster responsible for production of hyaluronidase, an important GBS virulence factor) (Granlund et al., 1998). A copy of IS1548 is also found downstream of the C5a peptidase gene (also associated with virulence), in isolates that contain it. Most IS1548-containing isolates were from patients with endocarditis and it was postulated that inactivation of hyaluronidase production and/or some effect on C5a peptidase may allow GBS isolates to adhere to and survive on heart valves.

We found IS1548 in all serosubtype III-1 isolates, which represented 52% of 58 serotype III isolates in our collection, from superficial (eight of 12) and normally sterile (22 of 46) specimens. The latter were from neonates (seven of 20), adults (three of six) and subjects of unspecified age (12 of 20) (data not shown). Although specific clinical data were unavailable, GBS endocarditis is uncommon and likely to have been present in few, if any, of these subjects. Further study is required to elucidate the association with this insertion sequence with specific virulence factors and clinical syndromes.

We found GBSi1, a group II intron, in 19% of our 224 isolates overall; it was commonly associated with IS861, and the distribution varied with serotype/serosubtype. It was rarely found in serotypes other than II and III. It was present in more than 50% of serotype II isolates, including four, which also contained IS1548. It was found in all serosubtypes III-2 and III-4 isolates, in which IS1548 was not found, but in no serosubtype III-1 isolates which did contain IS1548 or serosubtype III-3 isolates which did not.

Our subdivision of GBS serotype III into four serosubtypes, based on differences within the cps gene cluster was supported by corresponding differences in surface protein gene profiles and distribution of the five mobile elements described in this study. Although we did not test our isolates for hyaluronidase activity, it is likely that our serosubtype III-1, which expresses Rib protein and contains IS1548, IS861 and IS1381, corresponds with the hyaluronidase negative subtype III-2, described by Bohnsack et al., 2001. Our serosubtype III-2 also expresses Rib protein and contains IS861 and GBSi1 and probably corresponds with subtype III-3 of Bohnsack et al., 2001. Serosubtypes III-3 and III-4 were represented by relatively few isolates. The former (in common with some serotype Ia isolates) expressed the C alpha-like protein 2 and contained no mobile elements (an otherwise uncommon finding). The latter is closely related to serotype II, with which it shares sequence homology in a section of the cps gene cluster and various surface protein profiles and mobile elements.

### SUMMARY

Our aim has been to develop a comprehensive genotyping system for group B streptococcus (GBS). Such a system should ideally be reproducible, objective and transportable between laboratories, comparable with and complementary to other typing methods and able to incorporate known virulence markers. Based on these criteria, we first developed a molecular serotyping (MS) method based on the cps gene cluster. It compared favourably with, but was more sensitive than, conventional serotyping (CS) and allowed us to identify several subtypes of serotype (sst) III, as described by others. We have also developed a second molecular subtyping method based on the family of genes encoding variable surface protein antigens (bca/rib/alp2/alp3/alp4) and the IgA binding protein C beta (bac), is more sensitive and objective than conventional protein serotyping, which cannot type all isolates and is sometimes misleading. Our methods also can identify more members of the family of variable antigen genes and distinguish numerous bac subgroups. A third subtyping method uses five mobile genetic elements (mge) including four different insertion sequences (IS) and a type II intron, which have been identified in GBS. The use of this third method further enhances the discriminatory ability of our genotyping system.

We then used our typing system to examine the population genetic structure and age-related disease distribution of genotypes among 194 invasive GBS isolates.

We used mainly invasive GBS isolates to demonstrate the practical value of our genotyping system, confirm their clonal population structure and determine the distribution of genotypes in different patient groups. The isolates originated from patients of all ages with GBS sepsis. About half were consecutive GBS isolates from blood or CSF, at a large diagnostic laboratory in a general adult hospital, with an obstetric unit (i.e there were no isolates from children other than neonates). The rest were consecutive isolates referred for serotyping from all over New Zealand. Thus the overall age distribution is representative of that in the population affected by GBS disease, except that children beyond the early neonatal period are probably under-represented. However, the distribution of genotypes within each age-group should be representative.

Among our 194 Australasian invasive GBS isolates we identified 56 genotypes, of which five (Ia-1, Ib-1, III-1, III-2 and V-1) accounted for 62% of isolates.

The phylogenetic tree derived from our results showed relationships between cps serotype and protein gene profiles (pgp). Our results also show that certain known virulence markers—C beta, C alpha variants and hyaluronidase production (indirectly)—were associated with distinct clonal lineages.

Our genotyping system, based on three sets of genetic markers, is highly discriminatory. Because it provides useful phenotypic data, including antigenic composition, it will be useful for epidemiological surveillance of GBS, especially in relation to potential GBS vaccine use. Study of the relationships between putative high-virulence genotypes and patient characteristics (age and/or underlying risk factors), and whether there are significant differences between CSF isolates (or genotypes) and other invasive or colonising strains, will be facilitated by our genotyping system. Using this system, we have demonstrated a clonal population structure among invasive Australasian GBS isolates. This system will be applied to colonising GBS isolates, to identify markers of virulence.

Thus, we have developed an alternative to conventional serotyping for GBS, which is accurate and reproducible, can be performed by any laboratory with access to PCR/sequencing and, importantly, does not require panels of serotype-specific antisera that are increasingly difficult to maintain. All isolates are serotypable and sequencing of a relatively limited 790 bp region can provide additional serosubtyping information for MS III. The molecular methods we have described for serotype identification, together with the protein profiling (or protein antigen subtyping) and identification of mobile genetic elements (or mobile genetic elements subtyping) provide potentially useful markers for further phylogenetic and epidemiological studies of GBS as well as comprehensive strain identification that will be useful for epidemiological and other related studies that will be needed to monitor GBS isolates before and after introduction of GBS conjugate vaccines.

The various features and embodiments of the present, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are readily apparent to those skilled in molecular biology or related fields are intended to be within the scope of the following claims.

References

Ahmet, Z., P. Stanier, D. Harvey, and D. Holt. 1999. New PCR primers for the sensitive detection and specific identification of group B beta-hemolytic streptococci in cerebrospinal fluid. Mol. Cell. Probes. 13:349-357.

Arakere, G., A. E. Flores, P. Ferrieri, and C. E. Frasch. 1999. Inhibition enzyme-linked immunosorbent assay for serotyping of group B streptococcal isolates. J. Clin. Microbiol. 37:2564-2567.

Bohnsack, J. F., S. Takahashi, S. R. Detrick, L. R. Pelinka, L. L. Hammitt, A. Aly, A. A. Whiting, and E. E. Adderson. 2001. Phylogenetic Classification of Serotype III Group B Streptococci on the Basis of hylB Gene Analysis and DNA Sequences Specific to Restriction Digest Pattern Type III-3. J. Infect. Dis. 183:1694-1697.

Cropp, C. B., R. A. Zimmerman, J. Jelinkova, A. H. Auernheimer, R. A. Bolin, and B. C. Wyrick. 1974. Serotyping of group B streptococci by slide agglutination fluorescence microscopy, and microimmunodiffusion. J. Lab. Clin. Med. 84:594-603.

Granlund, M., L. Oberg, M. Sellin, and M. Norgren. 1998. Identification of a novel insertion element, IS1548, in group B streptococci, predominantly in strains causing endocarditis. J. Infect. Dis. 177:967-976

Hakansson, S., L. G. Burman, J. Henrichsen, and S. E. Holm. 1992. Novel coagglutination method for serotyping group B streptococci. J. Clin. Microbiol. 30:3268-3269.

Harrison, L. H., J. A. Elliott, D. M. Dwyer, J. P. Libonati, P. Ferrieri, L. Billmann, and A. Schuchat. 1998. Serotype distribution of invasive group B streptococcal isolates in Maryland: implications for vaccine formulation. Maryland Emerging Infections Program. J. Infect. Dis. 177:998-1002.

Hassan, A. A., A. Abdulmawjood, A. O. Yildirim, K. Fink, C. Lammler, and R. Schlenstedt. 2000. Identification of streptococci isolated from various sources by determination of cfb gene and other CAMP-factor genes. Can. J. Microbiol. 46:946-951.

Hickman, M. E., M. A. Rench, P. Ferrieri, and C. J. Baker. 1999. Changing epidemiology of group B streptococcal colonization. Pediatrics. 104:203-209.

Holm, S. E., and S. Hakansson. 1988. A simple and sensitive enzyme immunoassay for determination of soluble type-specific polysaccharide from group B streptococci. J. Immunol. Methods. 106:89-94.

Ke, D., C. Menard, F. J. Picard, M. Boissinot, M. Ouellette, P. H. Roy, and M. G. Bergeron. 2000. Development of conventional and real-time PCR assays for the rapid detection of group B streptococci. Clin. Chem. 46:324-331.

Kong, F., X. Zhu, W. Wang, X. Zhou, S. Gordon, and G. L. Gilbert. 1999. Comparative analysis and serovar-specific identification of the multiple banded antigen genes of Ureaplasma urealyticum biovar one. J. Clin. Microbiol. 37: 538-543.

Kong, F., S. Gordon, and G. L. Gilbert. 2000. Rapid-Cycle PCR for Detection and Typing of Mycoplasma pneumoniae in Clinical Specimens. J. Clin. Microbiol. 38:4256-4259.

Maeland, J. A., O. G. Brakstad, L. Bevanger, and A. I. Kvam. 1997. Streptococcus agalactiae beta gene and gene product variations. J. Med. Microbiol. 46:999-1005.

Maeland, J. A., O. G. Brakstad, L. Bevanger, and S. Krokstad. 2000. Distribution and expression of bca, the gene encoding the c alpha protein, by Streptococcus agalactiae. J. Med. Microbiol. 49:193-198.

Mawn, J. A., A. J. Simpson, and S. R. Heard. 1993. Detection of the C protein gene among group B streptococci using PCR. J. Clin. Pathol. 46:633-636.

Nagano, Y., N. Nagano, S. Takahashi, K. Murono, K. Fujita, F. Taguchi, and Y. Okuwaki. 1991. Restriction endonuclease digest patterns of chromosomal DNA from group B beta-haemolytic streptococci. J. Med. Microbiol. 35:297-303.

Rolland, K., C. Marois, V. Siquier, B. Cattier, and R. Quentin. 1999. Genetic features of Streptococcus agalactiae strains causing severe neonatal infections, as revealed by pulsed-field gel electrophoresis and hyIB gene analysis. J. Clin. Microbiol. 37:1892-1898.

Tamura, G. S., M. Herndon, J. Przekwas, C. E. Rubens, P. Ferrieri, and S. L. Hillier. 2000. Analysis of restriction fragment length polymorphisms of the insertion sequence IS 1381 in group B Streptococci. J. Infect. Dis. 181:364-368.

Triscott, M. X., and G. H. Davis. 1979. A comparison of four methods for the serotyping of group B streptococci. Aust. J. Exp. Biol. Med. Sci. 57:521-527.

Wilkinson, H. W., and M. D. Moody. 1969. Serological relationships of type I antigens of group B streptococci. J. Bacteriol. 97:629-34.

Zuerlein, T. J., B. Christensen, and R. T. Hall. 1991. Latex agglutination detection of group-B streptococcal inoculum in urine. Diagn. Microbiol. Infect. Dis. 14:191-194.

TABLE 1
GBS reference panels used in this study.

| | | | | GenBank |
|---|---|---|---|---|
| | | | MS/ | accession |
| Lab strain number | Source | Serotype | serosubtype | numbers |
| Reference panel 1¹ | | | | |
| 090 | Channing | la | la | AF332893 |
| H36B | Channing | lb | lb | AF332903 |
| 18RS21 | Channing | II | II | AF332905 |
| M781 | Channing | III | III-2³ | AF332896 |
| 3139 | Channing | IV | IV | AF332908 |
| CJB 111 | Channing | V | V | AF332910 |
| SS1214 | Channing | VI | VI | AF332901 |
| 7271 | Channing | VII | VII | AF332913 |
| JM9 130013 | Channing | VIII | VIII | |
| Reference panel 2² | | | | |
| NZRM 908 | ESR | la | la | AF332894 |
| (NCDC SS615) | | | | |
| NZRM 909 | ESR | lb | lb | AF332904 |
| (NCDC SS618) | | | | |
| NZRM 910 | ESR | lc | la | AF332914 |
| (NCDC SS700) | | | | |
| NZRM 911 | ESR | II | II | AF332906 |
| (NCDC SS619) | | | | |
| NZRM 912 | ESR | III | III-3³ | AF332897 |
| (NCDC SS620) | | | | |
| NZRM 2217 | ESR | Non-typable | II | AF332907 |
| (Prague 25/60) | | (R) | | |
| NZRM 2832 | ESR | IV | IV | AF332909 |
| (Prague 1/82) | | | | |
| NZRM 2833 | ESR | V | V | AF332911 |
| (Prague 10/84) | | | | |
| NZRM 2834 | ESR | VI | VI | AF332902 |
| (Prague 118754) | | | | |

Notes.
¹Reference panel 1: supplied by Dr Lawrence Paoletti, Channing Laboratory, Boston, USA.
²Reference panel 2: New Zealand Reference Medical Culture Collection strains supplied by Dr Diana Martin, ESR, Porirua, Wellington, New Zealand.
³MS III serosubtypes based on sequence heterogeneity; see text for more detail

TABLE 2
Oligonucleotide primers used in this study.

| | | | | |
|---|---|---|---|---|
| | | | GenBank | |
| | Target | | accession | |
| Primer | gene | Tm ° C.¹ | numbers | Sequence²⁻⁴ |

| | | | | |
|---|---|---|---|---|
| CFBS | cfb | 56.7 | X72754 | 328GAT GTA TCT ATC TGG AAC TCT AGT G352 |
| Sag59⁵ | cfb | 77.4 | X72754 | 350GTGGCTGGTGCATTGTTAT TTT CAC CAG CTG TAT |
| | | | | TAG AAG TA391 |
| Sag190⁵ | cfb | 76.8 | X72754 | 545CATTAACCGGTTTTTCATAATCT GTT CCC TGA ACA |
| | | | | TTA TCT TTG AT500 |
| CFBA | cfb | 63.2 | X72754 | 568TTT TTC CAC GCT AGT AAT AGC CTC545 |
| 16SS | 16S rRNA | 69.3 | AB023574 | 1441GCC GCC TAA GGT GGG ATA GAT G1462 |
| 23SA | 23S rRNA | 65.7 | X68427 | 70CGT CGT TTG TCA CGT CCT TC51 |
| DSF2⁶ | 16S rRNA | 75.9 | AB023574 | 975CATCCTTCTGACC GGC CTA GAG ATA GGC TTT |
| | | | | CT1007 |
| DSR1⁶ | 16S rRNA | 81.5 | AB023574 | 1250CGTCACCGG CTT GCG ACT CGT TGT ACC |
| | | | | AA1222 |
| cpsDS | cpsD | 69.1 | AB028896 (Ia), | 4892/4593GCA AAA GAA CAG ATG GAA CAA AGT |
| | | | AF163833 (III) | GG5007/4618 |
| cpsES | cpsE | 65.7 | AB028896 (Ia), | 5300/4910CTT TTG GAG TCG TGG CTA TCT |
| | | | AF163833 (III) | TG5322/4932 |
| cpsEA1 | cpsE | 65.4 | AB028896 (Ia), | 5431/5041GA/T/GA AAA AAG GAA AGT CGT GTC G/ATT |
| | | | AF163833 (III) | G5612/5017 |
| cpsES1 | cpsE | 65.9 | AB028896 (Ia), | 5612/5222CTT GGA C/TTC CTC TGA AAA GGA |
| | | | AF163833 (III) | TTG5635/5245 |
| cpsEA2 | cpsE | 66.8 | AB028896 (Ia), | 5723/5333AAA A/CGC TTG ATC AAC AGT TAA GCA |
| | | | AF163833 (III) | GG5698/5308 |
| cpsES2 | cpsE | 70.2 | AB028896 (Ia), | 6012/5622GAT GGT/C GGA CCG GCT ATC TTT TCT |
| | | | AF163833 (III) | C6036/5646 |
| cpsEA3 | cpsE | 63.7 | AB028896 (Ia), | 6116/5726CTT AAT TTG TTC TGC ATC TAC TCG |
| | | | AF163833 (III) | C6092/5702 |
| cpsES3 | cpsE | 71.5 | AB028896 (Ia), | 6410/6020GTT AGA TGT TCA ATA TAT CAA TGA ATG |
| | | | AF163833 (III) | GTC TAT TTG GTC AG6450/6060 |
| cpsEFA | CpsE/F | 62.1 | AB028896 (Ia), | 6526/6136CCT TTC AAA CCT TAC CTT TAC TTA |
| | spacer | | AF163833 (III) | GC6501/6111 |
| cpsFS | cpsF | 75.0 | AB028896 (Ia), | 6777/6387CAT CTG GTG CCG CTG TAG CAG TAC CAT |
| | | | AF163833 (III) | T6804/6414 |
| cpsFA | cpsF | 73.2 | AB028896 (Ia), | 6859/6469GTC GAA AAC CTC TAT A/GT A AAC/T GGT |
| | | | AF163833 (III) | CTT ACA A/GCC AAA TAA CTT ACC6819/6425 |
| cpsGA | cpsG | 54.7 | AB028896 (Ia), | 7162/6772AAG/C AGT TCA TAT CAT CAT ATG AGA G |
| | | | AF163833 (III) | 7138/6748 |
| cpsGA1 | cpsG | 74.5 | AB028896 (Ia), | 7199/6809CCG CCA/G TGT GTG ATA ACA ATC TCA GCT |
| | | | AF163833 (III) | TC7171/6781 |
| cpsGS | cpsG | 72.24 | AB028896 (Ia), | 7145/6755ATG ATG ATA TGA ACT CTT ACA TGA AAG |
| | | | AF163833 (III) | AAG CTG AGA TTG 7183/6793 |
| cpsGS1 | cpsG | 71.62 | AB028896 (Ia), | 7155/6765GAA CTC TTA CAT GAA AGA AGC TGA GAT |
| | | | AF163833 (III) | TGT TAT CAC AC 7192/6802 |
| IacpsHS | cpsH | 73.6 | AB028896 (Ia) | 7698CAT TCT TTG TTT AAA AA/CT CCT GAT TTT GAT |
| | | | | AGA ATT TTA GCA GC7741 |
| IacpsHA | cpsH | 75.2 | AB028896 (Ia) | 7993GAA TAT TCA AAA AAT CCC ATT GCT CTT TGA |
| | | | | GTA TGC ATA CC7953 |
| IacpsHA1 | cpsH | 66.4 | AB028896 (Ia) | 8271GTA AGT TAT CAA AAT ATA ACA TCA TTA CTA |
| | | | | TTA CTA GTA GAA ACG G8226 |
| IacpsHS1 | cpsH | 77.9 | AB028896 (Ia) | 8463GGC CTG CTG GGA TTA ATG AAT ATA GTT CCA |
| | | | | GGT TTG C8499 |
| IacpsHA2 | cpsH | 58.5 | AB028896 (Ia) | 8499GCA AAC CTG GAA CTA TAT TCA T8478 |
| IbcpsHS0 | cpsH | 58.6 | AB050723 (Ib) | 3013ATT GCT GCA TTC AAT TCA C3031 |
| IbcpsHS | cpsH | 81.9 | AB050723 (Ib) | 3016GCT GCA TTC AAT TCA CTG GCA GTA GGG GTT |
| | | | | GTG TCC3051 |
| IbcpsHA | cpsH | 67.7 | AB050723 (Ib) | 3297GAT AGT TAA GGG TAT TAT AAG ATT TGA ATA |
| | | | | TTC AAA GAA AGC3256 |
| IbcpsHS1 | cpsH | 74.1 | AB050723 (Ib) | 3546TTT GGT GAG CAT ATA TAA TAG AAT AAT CAA |
| | | | | TTT GCG GTC G3585 |
| IbcpsHS2 | cpsH | 73.7 | AB050723 (Ib) | 3740CTG GCC TAT TTG GAC TAA TAA ATG TGA TTT |
| | | | | TAG GTT TGT TTC3781 |
| IbcpsHA01 | cpsH | 57.7 | AB050723 (Ib) | 3781GAA ACA AAC CTA AAA TCA CAT TTA3758 |
| IbcpsHA1 | cpsH | 78.5 | AB050723 (Ib) | 3894GGC GCC ATC AAT ATC TTC AAG TGC AAA AAA |
| | | | | TGA AAA TAG G3855 |
| IbcpsIA | cpsI | 78.2 | A8050723 (Ib) | 4086CTA TCA ATG AAT GAG TCT GTT GTA GGA CGG |
| | | | | ATT GCA CG4049 |
| IbcpsIS | cpsI | 71.1 | AB050723 (Ib) | 4116GAT AAT AGT GGA GAA ATT TGT GAT AAT TTA |
| | | | | TCT CAA AAA GAC G4158 |
| IbcpsIA1 | cpsI | 78.6 | AB050723 (Ib) | 4638CCT GAT TCA TTG CAG AAG TCT TTA CGA TGC |
| | | | | GAT AGG TG4601 |
| IIIVIcpsHS | cpsH | 75.3 | AF163833 (III), | 7275/7120CAA GAG GAT ATA ACG TTT CAG CGA TTT |
| | | | AF337958 (VI) | ATT GCT GAG C7311/7156 |
| IIIcpsHS | cpsH | 72.1 | AF163833 (III) | 7672GAA TAC TAT TGG TCT GTA TGT TGG TTT TAT |
| | | | | TAG CAT CGC7710 |
| IIIcpsHA | cpsH | 71.0 | AF163833 (III) | 7817GTT ATA AGA AAA ACA AGCGGT GAT AAA TAA |
| | | | | GAA AGT CAT ACC7776 |
| IVcpsHS | cpsH | 74.1 | AF355776 (IV) | 7552CCG TAC ATA CAA CTG TTC TTG TTA GCA TTT |
| | | | | ACT TTT CTT TGC7593 |
| IVcpsHS1 | cpsH | 71.2 | AF355776 (IV) | 7887CCC AAG TAT AGT TAT GAA TAT TAG TTG GAT |
| | | | | GGT TTT TGG7925 |
| IVcpsHA | cpsH | 77.3 | AF355776 (IV) | 7951CAT CTA CAC CCC CAC AAA ATA TTT TCC CAA |
| | | | | AAA CCA TC7914 |
| IVcpsHA1 | cpsH | 58.7 | AF355776 (IV) | 7958TGT AAA TCA TCT ACA CCC CC 7939 |
| IVcpsMA | cpsM | 80.7 | AF355776 (IV) | 8265GGG TCA ATT GTA TCG TCG CTG TCA ACA AAA |
| | | | | CCA ATC AAA TC8225 |
| VcpsHS | cpsH | 76.3 | AF349539 (V) | 6943GGG TTT AGG CGA GGG AAA CTC AGC TTA CAA |
| | | | | AAT AGT G6979 |
| VcpsHS1 | cpsH | 72.2 | AF349539 (V) | 7258CAA TTT TTA TAG GGA TGG ACA ATT TAT TCT |
| | | | | GAG AAG TGA C7297 |
| VcpsHA | cpsH | 71.1 | AF349539 (V) | 7291TCT CAG AAT AAA TTG TCC ATC CCT ATA AAA |
| | | | | ATT GAC ATA C7252 |
| VcpsHS02 | cpsH | 59.0 | AF349539 (V) | 7616GAT GTT CTT TTA ACA GGT AGA TTA CAC7642 |
| VcpsHA1 | cpsH | 66.8 | AF349539 (V) | 7658GTT GTA AAT GAG CAT AGT GTA ATC TAC CTG |
| | | | | TTA AAA GAA C7619 |
| VcpsHS2 | cpsH | 74.0 | AF349539 (V) | 7871CCC AGT GTG GTA ATG AAT ATT AGT TGG CTA |
| | | | | GTT TTT GG7908 |
| VcpsHA2 | cpsH | 58.6 | AF349539 (V) | 7945CTT TTT TAT AGG TTC GAT ACC ATC7922 |
| VcpsMA | cpsM | 73.1 | AF349539 (V) | 8244CCC CCC ATA AGT ATA AAT AAT ATC CAA TCT |
| | | | | TGC ATA GTC AG8204 |
| VIcpsHS | cpsH | 76.7 | AF337958 (VI) | 7478CAC TAT TCC TAG TTT TTT GTG CAT ATT TGA |
| | | | | CAG GGG CAA G7517 |
| VIcpsHA | cpsH | 76.7 | AF337958 (VI) | 7517CTT GCC CCT GTC AAA TAT GCA CAA AAA ACT |
| | | | | AGG AAT AGT G7478 |
| VIcpsHS1 | cpsH | 77.2 | AF337958 (VI) | 7767CCT TAT TGG GCA AGG TAT AAG AGT TCC CTC |
| | | | | CAG TGT G7803 |
| VIcpsHA1 | cpsH | 77.2 | AF337958 (VI) | 7804CCA CAC TGG AGG GAA CTC TTA TAC CTT GCC |
| | | | | CAA TAA G7768 |
| VIcpsIA | cpsI | 74.5 | AF337958 (VI) | 8126GAA GCA AAG ATT CTA CAC AGT TCT CAA TCA |
| | | | | CTA ACT CCG8088 |
| cpsIA | cpsI | 70.3 | AB028896 (Ia), | 8816/8312GTA TAA CTT CTA TCA ATG GAT GAG TCT |
| | | | AF163833 (III) | GTT GTA GTA CGG8778/8274 |

Notes.
¹The primer Tm values are provided by the primer synthesiser (Sigma-Aldrich).
²Numbers represent the numbered base positions at which primer sequences start and finish (numbering start point “1” refer to the start points “1” of correspondent gene GenBank accession numbers).
³Underlined sequences show bases added to modify previously published primers.
⁴Letters behind “/” indicate alternative nucleotides in different serotypes.
⁵Ke et al., 2000.
⁶Ahmet et al., 1999

TABLE 3
Specificity and expected lengths of amplicons of using different
oligonucleotide primer pairs.

| | | Length of amplicons |
|---|---|---|
| Primer pairs* | Specificity | (base pairs) |
| Sag59/Sag190^{a} | GBS (S. agalactiae) | 196 |
| CFBS/CFBA | GBS (S. agalactiae) | 241 |
| 16SS/23SA | GBS (S. agalactiae) | 433 |
| DSF2/DSR1^{a} | GBS (S. agalactiae) | 276 |
| cpsDS/cpsEA1 | serotypes Ia to VII | 449/458 |
| cpsES/cpsEA2 | serotypes Ia to VII | 424 |
| cpsES1/cpsEA3 | serotypes Ia to VII | 505 |
| cpsES2/cpsEFA | serotypes Ia to VII | 515 |
| cpsES3/cpsFA^{b} | serotypes Ia to VII | 450 |
| cpsFS/cpsGA1^{b} | serotypes Ia to VII | 423 |
| cpsES3/cpsGA1^{b} | serotypes Ia to VII | 790 |
| cpsGS/cpslA | serotypes Ia and III | 1672/1558 |
| cpsGS1/cpslA | serotypes Ia and III | 1662/1548 |
| cpsGS/lacpsHA1 | serotype Ia | 1127 |
| cpsGS1/lacpsHA1 | serotype Ia | 1117 |
| lacpsHS/lacpsHA | serotype Ia | 296 |
| lacpsHS/lacpsHA1 | serotype Ia | 574 |
| lacpsHS1/cpslA^{c} | serotype Ia | 354 |
| cpsGS/lbcpsHA1 | serotype Ib | 1468 |
| cpsGS1/lbcpsHA1 | serotype Ib | 1458 |
| cpsGS/lbcpslA | serotype Ib | 1660 |
| cpsGS1/lbcpslA | serotype Ib | 1650 |
| lbcpsHS/lbcpsHA | serotype Ib | 282 |
| lbcpsHS1/lbcpsHA1 | serotype Ib | 349 |
| lbcpsHS2/lbcpslA | serotype Ib | 347 |
| lbcpslS/lbcpslA1^{c} | serotype Ib | 523 |
| cpsGS/IIIcpsHA | serotype III | 1063 |
| cpsGS1/IIIcpsHA | serotype III | 1053 |
| IIIVlcpsHS/IIIcpsHA | serotype III | 543 |
| IIIcpsHS/cpslA^{c} | serotype III | 641 |
| cpsGS/IVcpsHA | serotype IV | 1372 |
| cpsGS1/IVcpsHA | serotype IV | 1362 |
| cpsGS/IVcpsMA | serotype IV | 1686 |
| cpsGS1/IVcpsMA | serotype IV | 1676 |
| IVcpsHS/IVcpsHA | serotype IV | 400 |
| IVcpsHS1/IVcpsMA^{c} | serotype IV | 379 |
| cpsGS/VcpsHA1 | serotype V | 1096 |
| cpsGS1/VcpsHA1 | serotype V | 1086 |
| cpsGS/VcpsMA | serotype V | 1682 |
| CpsGS1/VcpsMA | serotype V | 1672 |
| VcpsHS/VcpsHA | serotype V | 349 |
| VcpsHS1/VcpsHA1 | serotype V | 401 |
| VcpsHS2/VcpsMA^{c} | serotype V | 374 |
| IIIVIcpsHS1/VIcpsHA | serotype VI | 398 |
| cpsGS/VIcpsHA1 | serotype VI | 1205 |
| cpsGS1/VIcpsHA1 | serotype VI | 1195 |
| cpsGS/VIcpslA | serotype VI | 1527 |
| cpsGS1/VIcpslA | serotype VI | 1517 |
| VIcpsHS/VIcpsHA1^{c} | serotype VI | 327 |
| VIcpsHS1/VIcpslA | serotype VI | 360 |

Notes.
*See Table 2 for primer sequences and FIG. 1 for some primer sites. Primers used in Algorithm for molecular serotype identification-FIG. 2
^{a}to identify GBS,
^{b}for sequencing,
^{c}for MS-specific PCR

TABLE 4

The heterogeneity of 8 GBS serotypes in the regions of the 3′-end of cpsD-cpsE-
cpsF-and the 5′-end of cpsG.

| | | | | | | | | | |
|---|---|---|---|---|---|---|---|---|---|
| Sites | Ia | Ib | II/III-4 | III | IV | V | VI | VII | Specificity |

| | | | | | | | | | |
|---|---|---|---|---|---|---|---|---|---|
| cpsD gene | | | | | | | | | |
| 62 | G | A | G⁴ | A | A | A | A | G | Ia, II, VII |
| 78-86 | −Ia-2¹; | − | −II-2^{2,4}; | −III-2³; | + | + | − | + | See text |
| repetitive sequence | +Ia-1¹ | | +II-1² | +III-1³, | | | | | |
| -TTACGGCGA | | | | III-3³ | | | | | |
| cpsD/cpsE genes | | | | | | | | | |
| spacer | | | | | | | | | |
| 138 | G | G | G | G | G | A⁵ | G | G | V |
| 139 | G | G | G | A III-2; | G | G | G | G | III-2 |
| | | | | G III-1, III-3 | | | | | |
| 144 | T | T | T | G III-2; | T | T | T | T | III-2 |
| | | | | T III-1, III-3 | | | | | |
| cpsE gene | | | | | | | | | |
| 198 | A | C | A⁴ | A | C | C⁵ | A | A | Ib, IV, V |
| 204 | G | G | G | A III-2, III-3; | G | G | G | G | III-2, III-3 |
| | | | | G III-1 | | | | | |
| 211 | T | T | T | T | T | T | G | T | VI |
| 218 | C | C | C | C | C | C | T | C | VI |
| 240 | T | T | T | T | T | T | C | T | VI |
| 249 | T | C | T⁴ | T | C | C⁵ | T | T | Ib, IV, V |
| 300 | C | C | C | T III-2; | C | C | C | C | III-2 |
| | | | | C III-1, III-3 | | | | | |
| 321 | C | C | C | T III-1; | C | C | C | C | III-1 |
| | | | | C III-2, III-3 | | | | | |
| 419 | T | C | T⁴ | T | T | T | T | T | Ib |
| 429 | A | T | A⁴ | T | T | T | T | A | Ia, II, VII |
| 437 | C | C | C; | C | C | C | C | T | VII, III-4 |
| | | | T III-4 | | | | | | |
| 457 | T | A | C⁴ | A | A | A | A | C | Ia, II, VII |
| 466 | G | G | G | G | A | G | G | A | IV |
| 486 | G | A | A | G III-3; | A | A | A | A | Ia, III-3 |
| | | | | A III-2, III-1 | | | | | |
| 602 | G | G | A⁴ | G | G | G | G | A | II, VII |
| 606 | T | T | T | T | T | T | C | T | VI |
| 627 | T | C | C | C | C | C | C | C | Ia |
| 636 | C | T | T | C III-1; | T | T | T | T | Ia, III-1 |
| | | | | T III-2, III-3 | | | | | |
| 645 | C | T | C⁴ | C | T | T | C | C | Ib, IV, V |
| 803 | A | A | A | A | A | A | T | A | VI |
| 971 | C | T | T | C | C | C | T | T | Ia, III, IV, V |
| 1026 | A | G | G | G III-2, III-1; | A | A | G | G | Ia, III-3, IV, V |
| | | | | A III-3 | | | | | |
| 1044 | T | T | T | T | T | T | C | T | VI |
| 1173 | A | G | A | A | A | A | A | A | Ib |
| 1194 | C | C | C | A | A | C | A | C | III, IV, VI |
| 1251 | G | G | G | G | G | G | A | G | VI |
| 1278 | A | A | A | A | A | G | A | A | V |
| 1413 | C | T | T | C III-3; | T | T | T | T | Ia, III-3 |
| | | | | T III-2, III-1 | | | | | |
| 1495 | C | C | C | C | C | C | A | C | VI |
| 1500 | A | G | A | A | A | A | A | A | Ib |
| 1501 | C | C | T | C | C | C | C | T | II, VII |
| 1512 | C | T | C | T III-2, III-1; | C | T | T | C | Ia, II, III-3, IV, VII |
| | | | | C III-3 | | | | | |
| 1518 | T | C | T | C III-2, III-1; | T | C | C | T | Ia, II, III-3, IV, VII |
| | | | | T III-3 | | | | | |
| 1527 | T | A | A | T III-3; | T | A | A | A | Ia, III-3, IV |
| | | | | A III-2, III-1 | | | | | |
| cpsF gene | | | | | | | | | |
| 1595 | T | C | T | T | T | T | C | T | Ib, VI |
| 1611 | C | C | C | C | C | C | C | T | VII |
| 1620 | C | C | C | C | C | C | C | T | VII |
| 1627 | G | G | G | G | T | G | G | G | IV |
| 1629 | G | G | G | A III-1; | G | G | G | G | III-1 |
| | | | | G III-2, III-3 | | | | | |
| 1655 | C | T | C | C | C | C | C | C | Ib |
| 1832 | C | C | C | C | T | C | C | C | IV |
| 1856 | T | C | T | T | T | T | T | T | Ib |
| 1866 | G | G | G | G | G | G | G | A | VII |
| 1871 | T | T | T | T | T | C | T | T | V |
| 1892 | A | A | A | A | A | G | A | A | V |
| 1971 | G | G | G | G | G | G | A | G | VI |
| cpsG gene | | | | | | | | | |
| 2026 | G | A | G | G | G | G | G | G | Ib |
| 2088 | G | G | G | G | A | G | G | G | IV |
| 2134 | T | T | T | C III-2, III-1; | T | T | T | T | III-2, III-1 |
| | | | | T III-3 | | | | | |
| 2187 | C | C | C | C | C | C | C | G | VII |
| 2196 | A | A | A | A | A | A | A | G | VII |

Notes.
¹Repetitive sequence: serosubtype Ia-1 present (+); serosubtype Ia-2 absent (−) (see text).
²Repetitive sequence: serosubtype II-1 present (+); serosubtype II-2 absent (−) (see text).
³Repetitive sequence: serosubtypes III-1 and III-3 present (+); serosubtype III-2 absent (−); serosubtype III-4 variable (see text)
⁴One CS II strain has mutations at the 9 sites (see text).
⁵At positions 138, 198, and 249, one CS V reference strain (Prague 10/84) is identical with corresponding sequence in GenBank (GenBank accession number AF349539), the sequences are G, A and T, respectively; another CS V reference strain (CJB 111) and all the other sequenced CS V strains are identical, the sequences are A, C and C, respectively.

TABLE 5
Comparison of the results of conventional serotyping (CS) and
molecular serotype identification (MS)/subtyping of 206 clinical GBS
isolates.

| | MS/serosubtype | | | | | | | | | | |
|---|---|---|---|---|---|---|---|---|---|---|---|
| CS | Ia | Ib | II | III-1¹ | III-2¹ | III-3¹ | III-4¹ | IV | V | VI | VIII |
| Ia | 38 | | | | | | | | | | |
| Ib | | 30 | | | | | | | | | |
| II | | | 25 | | | | | | | | |
| III | | | | 27 | 20 | 4 | 3 | | | | |
| IV | | | | | | | | 7 | | | |
| V | | | | | | | | | 31 | | |
| VI | | | | | | | | | | 2 | |
| VIII | | | | | | | | | | | 1 |
| NT¹ | 2 | 5 | 1 | 3 | 1 | | | | 5 | 1 | |
| Total | 40 | 35 | 26² | 30 | 21² | 4 | 3 | 7 | 36 | 3 | 1 |
| (206)² | | | | | | | | | | | |

Notes.
¹For details of MS III serosubtypes see text.
²One mixed culture was included as two separate isolates (one serotype II, one subtype III-2).

TABLE 6

Oligonucleotide primers used in this study.

| | | | | |
|---|---|---|---|---|
| | | | GenBank | |
| | | | Accession | |
| Primer | Target gene | Tm ° C.¹ | numbers | Sequence^{2,3} |

| | | | | |
|---|---|---|---|---|
| IgAagGBS⁵ | bac | 73.8 | X59771 | 2663GCGATTAAACAA CAA ACT ATT TTT GAT A TTG |
| | | | | ACA ATG CAA2702 |
| IgAS1⁴ | bac | 72.8 | X59771 | 2765GCT AAA TTT CAA AAA GGT CTA GAG ACA AAT |
| | | | | ACG CCA G2801 |
| IgAA1⁴ | bac | 78.9 | X59771 | 3157CCC ATC TGG TAA CTT CGG TGC ATC TGG AAG |
| | | | | C3127 |
| RigAagGBS⁵ | bac | 76.3 | X59771 | 3284CAGCCAACTCTTTC GTC GTT ACT TCC TTG AGA |
| | | | | TGT AAC3247 |
| GBS1360S⁶ | bac | 72.3 | X59771 | 1325GTGAAATTGTAT AAG GCT ATG AGT GAG AGC TTG |
| | | | | GAG1360 |
| GBS1717S⁴ | bac | 75.0 | X59771 | 1685ACA GTC ACA GCT AAA AGT GAT TCG AAG ACG |
| | | | | ACG1717 |
| GBS1937A⁶ | bac | 75.9 | X59771 | 1976CCGTTTTAGAATCTTT CTG CTC TGG TGT TTT AGG |
| | | | | AAC TTG1937 |
| BcaRUS⁷ | bca repetitive unit | 73.5 | M97256 | 769GATAAATATGATCCAA CAG GAG GGG AAA CAA CAG |
| | | | | TAC805 |
| BCaRUA⁷ | bca repetitive unit | 77.2 | M97256 | 1003CTGGTTTTGGTGTCACAT GAA CCG TTA CTT CTA |
| | | | | CTG TAT CC963 |
| bcaS1⁴ | bca/alp2/alp3 | 71.7 | M97256 and | 208/533GGT AAT CTT AAT ATT TTT GAA GAG TCA ATA |
| | | | AF291065 | GTT GCT GCA TCT AC251/576 |
| bcaS2⁴ | bca/alp2/alp3 | 78.0 | M97256 and | 256/581CCAGGGA GTG CAG CGA CCT TAA ATA CAA |
| | | | AF291065 | GCA TC288/613 |
| bcaS⁴ | bca | 58.9 | M97256 | 370GTT TTA GAA CAA GGT TTT ACA GC392 |
| baIS⁴ | alp2/alp3 | 73.8 | AF291065 | 677GAT CCT CAA AAC CTC ATT GTA TTA AAT CCA TCA |
| | | | | AGC TAT TC717 |
| bcaA⁴ | bca | 74.2 | M97256 | 597CGTTCTAACTT CTT CAA TCT TAT CCC TCA AGG |
| | | | | TTG TTG560 |
| baIA⁴ | alp2/alp3 | 73.6 | AF291065 | 978CCA GTT AAG ACT TCA TCA CGA CTC CCA TCA |
| | | | | C948 |
| baI23S1⁴ | alp2/alp3 | 70.9 | AF208158 and | 1093/1373CAG ACT GTT AAA GTG GAT GAA GAT ATT |
| | | | AF291065 | ACC TTT ACG G1129/1409 |
| baI23S2⁴ | alp2/alp3 | 72.9 | AF208158 and | 1174/1454CTT AAA GCT AAG TAT GAA AAT GAT ATC |
| | | | AF291065 | ATT GGA GCT CGT G1213/1493 |
| baI2S⁴ | alp2 | 59.2 | AF208158 | 1363GTT CTT CCG CCA GAT AAA ATT AAG1386 |
| baI2A⁴ | alp2 | 58.3 | AF208158 | 1576CTG TTG ACT TAT CTG GAT AGG TC1554 |
| baI2A1⁴ | alp2 | 78.3 | AF208158 | 1426CGT GTT GTT CAA CAG TCC TAT GCT TAG CCT |
| | | | | CTG GTG1391 |
| baI2A2⁴ | alp2 | 70.8 | AF208158 | 1518GGT ATC TGG TTT ATG ACC ATT TTT CCA GTT ATA |
| | | | | CG1484 |
| baI3S⁴ | alp3 | 57.1 | AF291065 | 1643GTT CTT CCG CTT AAG GAT AGC A1664 |
| baI3A⁴ | alp3 | 79.2 | AF291065 | 1693GAC CGT TTG GTC CTT ACC TTT TGG TTC GTT |
| | | | | GCT ATC C1657 |
| #ribS1⁴ | rib | 65.2 | U58333 | 216TAC AGA TAC TGT GTT TGC AGC TGA AG241 |
| ribS2⁴ | rib | 73.0 | U58333 | 238GAAGTAATTTCAG GAA GTG CTG TTA CGT TAA ACA |
| | | | | CAA ATA TG279 |
| ribA1⁴ | rib | 78.8 | U58333 | 431GAA GGT TGT GTG AAA TAA TTG CCG CCT TGC |
| | | | | CTA ATG396 |
| ribA2⁴ | rib | 72.6 | U58333 | 462AAT ACT AGC TGC ACC AAC AGT AGT CAA TTC AGA |
| | | | | AGG427 |
| #ribA3⁴ | rib | 61.3 | U58333 | 570CAT CTA TTT TAT CTC TCA AAG CTG AAG554 |

Notes.
#For sequencing use only, not entirely specific for rib gene.
¹The primer Tm values are provided by the primer synthesiser (Sigma-Aldrich).
²Numbers represent the numbered base positions at which primer sequences start and finish (numbering start point “1” refer to the start point “1” of corresponding GenBank accession number, of which there are two for some sequences).
³Underlined sequences show bases added to modify previously published primers.
⁴Primers designed by us for this study.
⁵Mawn et al., 1993.
⁶Maeland et al., 1997.
⁷Maeland et al., 2000.

TABLE 7
Specificity and expected lengths of amplicons of using different
primer pairs.

| | | Length of | |
|---|---|---|---|
| | | amplicons | Protein profile |
| Primer pairs* | Specificity | (base pairs) | code |
| IgAagGBS/ | bac | 532-838 | B |
| RIgAagGBS | | | |
| IgAS1/IgAA1 | bac | 303-591 | B |
| GBS1360S/ | bac | 652 | B |
| GBS1937A | | | |
| GBS1717S/ | bac | 292 | B |
| GBS1937A | | | |
| bcaS1/bcaA | 5′-end of bca | 390 | A |
| bcaS2/bcaA | 5′-end of bca | 342 | A |
| BcaRUS/bcaRUA | bca repetitive unit/ | 235 | a/as |
| | bca repetitive unit-like | | |
| | region | | |
| bcaS1/balA | alp2/alp3 | 446 | alp2 or alp3 |
| bcaS2/balA | alp2/alp3 | 398 | alp2 or alp3 |
| balS/balA | alp2/alp3 | 302 | alp2 or alp3 |
| bal23S1/bal2A1 | alp2 | 334 | alp2 |
| bal23S2/bal2A1 | alp2 | 253 | alp2 |
| bal23S1/bal2A2 | alp2 | 426 | alp2 |
| bal23S2/bal2A2 | alp2 | 345 | alp2 |
| bal23S1/bal3A | alp3 | 321 | alp3 |
| bal23S2/bal3A | alp3 | 240 | alp3 |
| #ribS1/ribA3 | rib/rib-like | 355 | R/r |
| ribS2/ribA1 | rib | 194 | R |
| ribS2/ribA2 | rib | 225 | R |
| ribS2/ribA3 | rib | 333 | R |

Notes.
*See Table 6 for primer sequences.
#For sequencing use only, not entirely specific for rib gene (see text for more detail).

TABLE 8
Genetic groups and subgroups of bac gene (C beta protein gene)
based on amplicon length (using primers IgAagGBS/RIgAagGBS) and
sequence heterogeneity.

| | | | | No. of | |
|---|---|---|---|---|---|
| | | | | different | |
| | | | | sites | |
| | | | | compared | |
| | | | GenBank | with | Molecular |
| Group or | | Amplicon | accession | (c.f.) main | serotype/ |
| Subgroup | N = | length | numbers | group | serosubtypes |
| B1 | 19 | 532 | X58470 | | 17 = Ib; 2 = II |
| B1a | 1 | 532 | AF362686 | 1 (c.f. B1) | Ib |
| B2 | 3 | 550 | AF362687 | | Ib, II, III-4 |
| B3 | 2 | 586 | AF362688 | | 2 = Ib |
| B3a | 1 | 586 | AF362689 | 4 (c.f. B3) | V |
| B3b | 1 | 586 | AF362690 | 21 (c.f. B3) | VI |
| B3c | 1 | 586 | AF362691 | 24 (c.f. B3) | Ib |
| B4 | 8 | 604 | AF362692 | | 4 = Ib; 4 = II |
| B4a | 1 | 604 | AF362693 | 1 (c.f. B4) | II |
| B4b | 2 | 604 | AF362694 | 2 (c.f. B4) | 2 = Ib |
| B5 | 2 | 622 | AF362695 | | Ia, VI |
| B5a | 1 | 622 | AF362696 | 2 (c.f. B5) | Ia |
| B6 | 1 | 640 | AF362697 | | Ib |
| B7 | 1 | 658 | AF362698 | | Ib |
| B7a | 1 | 658 | AF362699 | 34 (c.f. B7) | VI |
| B8 | 1 | 712 | AF362700 | | Ib |
| B9 | 2 | 748 | AF362701 | | 2 = II |
| B9a | 1 | 748 | AF362702 | 13 (c.f. B9) | Ib |
| B10 | 2 | 820 | AF362703 | | 2 = Ib |
| B11 | 1 | 838 | AF362704 | | Ib |

Note.
*See Table 9 for further details of serotype/serosubtype relationships with protein antigens.

TABLE 9
The relationship between GBS protein gene profiles and capsular
polysaccharide (cps) molecular serotypes/serosubtypes.

| Serotype/ | | | | | | | | | | | |
|---|---|---|---|---|---|---|---|---|---|---|---|
| serosubtype* | N = | None | Aa | AaB | R | alp 3 | a | as | alp2as | RB | Ra |
| Ia | 43 | — | — | 2 | — | — | 35 | 3 | 3 | — | — |
| Ib | 37 | — | 1 | 35 | — | 1 | — | — | — | — | — |
| II | 29 | — | 3 | 10 | 8 | 2 | 5 | — | — | — | 1 |
| III-1 | 30 | — | — | — | 30 | — | — | — | — | — | — |
| III-2 | 22 | — | — | — | 22 | — | — | — | — | — | — |
| III-3 | 5 | — | — | — | — | — | — | — | 5 | — | — |
| III-4 | 3 | — | — | 1 | — | 1 | — | — | 1 | — | — |
| IV | 9 | — | — | — | 1 | — | 8 | — | — | — | — |
| V | 38 | 1 | — | — | 1 | 35 | — | — | — | 1 | — |
| VI | 5 | — | 1 | 3 | — | — | 1 | — | — | — | — |
| VII | 1 | — | — | — | — | 1 | — | — | — | — | — |
| VIII | 2 | 1 | — | — | — | 1 | — | — | — | — | — |
| Total | 224 | 2 | 5 | 51 | 62 | 41 | 49 | 3 | 9 | 1 | 1 |

Note.
*See text for explanation of cps serosubtypes and Table 7 for explanation of protein antigen gene profile codes.

TABLE 10

Oligonucleotide primers used in this study.

| | | | | |
|---|---|---|---|---|
| | | | GenBank | |
| | | | accession | |
| Primer | Target | Tm ° C.¹ | numbers | Sequence² |

| | | | | |
|---|---|---|---|---|
| IS861S | IS861 | 77.4 | M22449 | 445GAG AAA ACA AGA GGG |
| | | | | AGA CCG AGT AAA ATG GGA |
| | | | | CG479 |
| IS861A1 | IS861 | 77.3 | M22449 | 831CAC GAT TTC GCA GTT |
| | | | | CTA AAT AAA TCC GAC GAT |
| | | | | AGC C795 |
| IS861A2 | IS861 | 76.1 | M22449 | 1020CAA ACT CCG TCA CAT |
| | | | | CGG TAT AGC ACT TCT CAT |
| | | | | AGG985 |
| IS1548S | IS1548 | 76.5 | Y14270 | 143CTA TTG ATG ATT GCG |
| | | | | CAG TTG AAT TGG ATA GTC |
| | | | | GTC178 |
| IS1548S1 | IS1548 | 77.0 | Y14270 | 539GTT TGG GAC AGG TAG |
| | | | | CGG TTG AGG AGA AAA GTA |
| | | | | ATG574 |
| IS1548A1 | IS1548 | 77.0 | Y14270 | 574CAT TAC TTT TCT CCT |
| | | | | CAA CCG CTA CCT GTC CCA |
| | | | | AAC539 |
| IS1548A2 | IS1548 | 70.3 | Y14270 | 915CCC AAT ACC ACG TAA |
| | | | | CTT ATG CCA TTT G888 |
| IS1548A3 | IS1548 | 78.0 | Y14270 | 930CGT GTT ACG AGT CAT |
| | | | | CCC AAT ACC ACG TAA CTT |
| | | | | ATG CC893 |
| IS1381S1 | IS1381 | 80.1 | AF064785/ | 272/818CTT ATG AAC AAA |
| | | | AF367974 | TTG CGG CTG ATT TTG GCA |
| | | | | TTC ACG307/853 |
| IS1381S2 | IS1381 | 81.7 | AF064785/ | 497/1040GGC TCA GGC GAT |
| | | | AF367974 | TGT CAC AAG CCA AGG |
| | | | | GAG526/1069 |
| IS1381A | IS1381 | 73.1 | AF064785/ | 881/1424CTA AAA TCC TAG |
| | | | AF367974 | TTC ACG GTT GAT CAT TCC |
| | | | | AGC849/1392 |
| ISSa4S | ISSa4 | 78.5 | AF165983 | 326CGT ATC TGT CAC TTA |
| | | | | TTT CCC TGC GGG TGT CTC |
| | | | | C359 |
| ISSa4A1 | ISSa4 | 75.2 | AF165983 | 639GCC GAT GTC ACA ACA |
| | | | | TAG TTC AGG ATA TAG CCA |
| | | | | G606 |
| ISSa4A2 | ISSa4 | 74.5 | AF165983 | 780CGT AAA GGA GTC CAA |
| | | | | AGA TGA TAG CCT TTT TGA |
| | | | | ACC745 |
| GBSi1S1 | GBSi1 | 78.6 | AJ292930 | 721CAT CTC GGA ACA ATA |
| | | | | TGC TCG AAG CTT ACA AGC |
| | | | | AAG TG758 |
| GBSi1S2 | GBSi1 | 77.3 | AJ292930 | 789GGG GTC ACT ATC GAG |
| | | | | CAG ATG GAT GAC TAT CTT |
| | | | | CAC824 |
| GBSi1A1 | GBSi1 | 83.9 | AJ292930 | 1058AAT GGC TGT TTC GCA |
| | | | | GGA GCG ATT GGG TCT GAA |
| | | | | CC1024 |
| GBSi1A2 | GBSi1 | 80.5 | AJ292930 | 1161CCA GGG ACA TCA ATC |
| | | | | TGT CTT GCG GAA CAG TAT |
| | | | | CG1127 |

Notes.
¹The primer Tm values were provided by the primer synthesiser (Sigma-Aldrich).
²Numbers represent the numbered base positions at which primer sequences start and finish (numbering start point “1” refers to the start point “1” of corresponding gene GenBank accession number).

TABLE 11
Specificity and expected lengths of amplicons of using different
oligonucleotide primer pairs.

| | | Length of amplicons |
|---|---|---|
| Primer pairs* | Specificity | (base pairs) |
| IS861S/IS861A1 | IS861 | 387 |
| IS861S/IS861A2 | IS861 | 576 |
| IS1548S/IS1548A1 | IS1548 | 432 |
| IS1548S/IS1548A2 | IS1548 | 773 |
| IS1548S/IS1548A3 | IS1548 | 788 |
| IS1548S1/IS1548A2 | IS1548 | 377 |
| IS1548S1/IS1548A3 | IS1548 | 392 |
| IS1381S1/IS1381A | IS1381 | 610/607# |
| IS1381S2/IS1381A | IS1381 | 385 |
| ISSa4S/ISSa4A1 | ISSa4 | 314 |
| ISSa4S/ISSa4A2 | ISSa4 | 455 |
| GBSi1S1/GBSi1A1 | GBSi1 | 338 |
| GBSi1S1/GBSi1A2 | GBSi1 | 441 |
| GBSi1S2/GBSi1A1 | GBSi1 | 270 |
| GBSi1S2/GBSi1A2 | GBSi1 | 373 |

Notes.
*See table 10 for primer sequences.
#Our sequencing result (GenBank accession number: AF367974) was 3 bp shorter than that previously described by Tamura et al., 2000 (GenBank accession number: AF064785).

TABLE 12
Relationship between mobile genetic elements and capsular
polysaccharide serotypes, serotype III subtypes and surface protein gene
profiles.

| | | | | | | | | No |
|---|---|---|---|---|---|---|---|---|
| Serotype/ | Protein | | | | | | | mobile |
| serosubtype | gene profile | N = | IS861 | IS1548 | IS1381 | ISSa 4 | GBSi1 | element |
| Ia | AaB | 2 | 2 | — | 2 | — | — | — |
| Ia | alp2as | 3 | — | — | — | — | — | 3 |
| Ia | a | 35 | 3 | 1 | 35 | 1 | — | — |
| Ia | as | 3 | — | — | 3 | — | — | — |
| subtotal | | 43 | 5 | 1 | 40 | 1 | — | 3 |
| Ib | Aa | 1 | — | — | — | — | — | 1 |
| Ib | AaB | 35 | 30 | — | 35 | 1 | — | — |
| Ib | alp3 | 1 | — | — | 1 | — | — | — |
| subtotal | | 37 | 30 | — | 36 | 1 | — | 1 |
| II | Aa | 3 | 3 | 1 | 3 | 2 | 1 | — |
| II | AaB | 10 | 10 | 5 | 10 | 5 | 1 | — |
| II | alp3 | 2 | 1 | 1 | 2 | — | — | — |
| II | R | 8 | 8 | — | 8 | — | 8 | — |
| II | Ra | 1 | 1 | — | — | — | 1 | — |
| II | a | 5 | 2 | 2 | 5 | 3 | 5 | — |
| subtotal | | 29 | 25 | 9 | 28 | 10 | 16 | — |
| III-1 | R | 30 | 30 | 30 | 30 | 1 | — | — |
| III-2 | R | 22 | 22 | — | — | — | 22 | — |
| III-3 | alp2as | 5 | — | — | — | — | — | 5 |
| III-4 | AaB | 1 | 1 | — | 1 | — | 1 | — |
| III-4 | alp2as | 1 | — | — | — | — | 1 | — |
| III-4 | alp3 | 1 | — | — | 1 | | 1 | — |
| subtotal | | 60 | 53 | 30 | 32 | 1 | 25 | 5 |
| IV | R | 1 | 1 | — | 1 | — | 1 | — |
| IV | a | 8 | 2 | — | 8 | — | — | — |
| subtotal | | 9 | 3 | — | 9 | — | 1 | — |
| V | alp3 | 35 | 3 | 1 | 35 | 1 | 1 | — |
| V | R | 1 | 1 | — | 1 | 1 | — | — |
| V | RB | 1 | 1 | — | 1 | — | — | — |
| V | none | 1 | — | — | — | — | — | 1 |
| subtotal | | 38 | 5 | 1 | 37 | 1 | 1 | 2 |
| VI | Aa | 1 | — | — | 1 | — | — | — |
| | AaB | 3 | 3 | — | 3 | — | — | — |
| | a | 1 | — | — | — | 1 | 13 | — |
| subtotal | | 5 | 3 | — | 5 | — | — | — |
| VII | alp3 | 1 | — | — | 1 | — | — | — |
| VIII | alp3 | 1 | — | — | 1 | — | — | — |
| | none | 1 | — | — | 1 | — | — | — |
| subtotal | | 2 | — | — | 2 | — | — | — |
| Total | | 224 | 124 | 41 | 190 | 15 | 43 | 10 |
| | | | (55) | (18) | (85) | (7) | (19) | (4) |

Note.
A: 5′-end of bca gene (C alpha protein);
a: bca gene repetitive unit or bca gene repetitive unit-like sequence (multiple band amplicon);
as: bca gene repetitive unit or bca gene repetitive unit-like sequence (single band amplicon);
B: C beta/IgA binding protein (bac) gene.
R: Rib protein (rib) gene;
alp2: C alpha-like protein 2 (alp2) gene;
alp3: C alpha-like protein 3 (alp3) gene;
r: assumed Rib-like protein gene.

TABLE 13
Distribution of mobile genetic elements among 194 invasive
GBS isolates.
Mobile genetic elements present

| Total N = | IS1381 | IS861 | IS1548 | ISSa4 | GBSi1 | None |
|---|---|---|---|---|---|---|
| 6 | — | — | — | — | — | 6 |
| 78 | 78 | — | — | — | — | — |
| 2 | — | — | — | — | 2 | — |
| 37 | 37 | 37 | — | — | — | — |
| 1 | 1 | — | 1 | — | — | — |
| 3 | 3 | — | — | 3 | — | — |
| 29 | 29 | 29 | 29 | — | — | — |
| 6 | 6 | 6 | — | 6 | — | — |
| 8 | 8 | 8 | — | — | 8 | — |
| 18 | — | 18 | — | — | 18 | — |
| 1 | 1 | — | — | — | 1 | — |
| 1 | 1 | — | 1 | — | 1 | — |
| 2 | 2 | 2 | 2 | — | 2 | — |
| 2 | 2 | — | — | 2 | 2 | — |
| Total | 168 | 100 | 33 | 11 | 34 | 6 |
| (n = 194) | (87%) | (52%) | (17%) | (6%) | (18%) | (3%) |

Note.
Data are numbers of isolates containing various combinations of mge

TABLE 14
Relationship between GBS genotypes and invasive disease age.

| Serotype | Age-group/disease¹ | | | | | | |
|---|---|---|---|---|---|---|---|
| Genotype | 0-6 d | 7-3 m | 4 m-14 yr | 15-45 yr | 46-60 yr | >60 yr | Total |
| Ia-1 | 14 | 4 + 1 | 1 | 7 | 3 | 6 | 35 + 1 (19%) |
| Ia-(2-8) | 4 | 2 | — | 1 | — | 3 | 10 |
| Ia total | 18 (34%) | 6 + 1 (21%) | 1 (10%) | 8 (28%) | 3 (18%) | 9 (17%) | 45 + 1 (24%) |
| Ib-1 | 2 | 1 + 1 | — | 3 | 2 | 5 + 1 | 13 + 2 |
| Ib-(2-16) | 3 | 4 + 2 | — | 3 | 1 | 5 | 16 + 2 |
| Ib total | 5 (9.4%) | 5 + 3 (24%) | — | 6 (21%) | 3 | 10 + 1 | 29 + 4 (17%) |
| II | 8 (15%) | 1 (3%) | — | 4 + 1 (17%) | 1 | 4 (7%) | 18 + 1 (10%) |
| III-1 | 6 + 1 (13%) | 4 (12%) | 1 + 1 (20%) | 1 + 1 (7%) | 6 + 1 (41%) | 4 | 22 + 4 (13%) |
| III-2 | 5 (9%) | 5 + 4 (39%)³ | 1 (10%) | 2 | — | — | 13 + 4 (9%) |
| III-(3-4) | 1 + 1 | 1 | — | 1 | 1 | 1 | 5 + 1 |
| III total | 12 + 2 (26%) | 10 + 4 (41%) | 2 + 1 (30%) | 4 + 1 (17%) | 7 + 1 (44%) | 5 (9%) | 40 + 9 (25%) |
| IV total | 3 | — | — | — | — | 4 | 7 (4%) |
| V-1 | 3 | 3 | 2 | 4 | 2 | 13 + 1 | 27 + 1 (14%) |
| V-(2-7) | 1 | 1 | — | 1 | — | 4 | 7 |
| V total | 4 (8%) | 4 (12%) | 2 (20%) | 5 (17%) | 2 (11%) | 17 + 1 (33%)⁴ | 34 + 1 (18%) |
| VI total | 1 | — | — | — | +1 | 3 | 4 + 1 (3%) |
| TOTAL | 51 + 2 = 53 | 26 + 8 = 34 | 5 + 2 = 7 | 27 + 1 = 29 | 16 + 2 = 18 | 52 + 2 = 54 | 177 + 17 = 194 |

Notes:
¹Numbers after “+” refer to CSF isolates; all others are from blood.
²Five aged 4 m-1 yr and one case was aged 3 yr.
³Sst III-2 in late onset infection compared with all other groups: p = 0.0005, odds ratio (OR) 6.8; 95% confidence interval (CI) 2.4-19.4.
MS-V in elderly compared with all other age-groups: p = 0.001, OR 0.28; 95% CI 0.13-0.59).

The invention is further defined by the following numbered paragraphs:

1. A method of typing a group B streptococcal bacterium which method comprises analysing the nucleotide sequence of one or more regions within the cpsD, cpsE, cpsF. cpsG and/or cpsI/M genes of said bacterium, said region(s) comprising one or more nucleotides whose sequence varies between types.

2. A method according to paragraph 1 wherein the nucleotide sequence is analysed for one or more positions corresponding to positions 62, 78-86, 138, 139, 144, 198, 204, 211, 281, 240, 249, 300, 321, 419, 429, 437, 457, 466, 486, 602, 606; 627, 636, 645, 803, 971, 1026, 1044, 1173, 1194, 1251, 1278, 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

3. A method according to paragraph 1 wherein at least one region is within a sequence delineated by the 3′ 136 bases of the cpsE gene and the 5′ 218 bases of the cpsG gene of the cpsE-cpsF-cspG gene cluster of said streptococcal bacterium.

4. A method according to paragraph 3 wherein the nucleotide sequence is analysed for one or more positions corresponding to positions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

5. A method according to any one of paragraphs 1 to 4 wherein at least one region is within the cpsI/M genes of said bacterium.

6. A method according to any one of paragraphs 1 to 5 wherein the nucleotide sequence analysis step comprises sequencing said one or more regions.

7. A method according to any one of paragraphs I to 5 wherein the nucleotide sequence analysis step comprises determining whether a polynucleotide obtained from said bacterium selectively hybridises to a polynucleotide probe comprising one or more of the said regions.

8. A method according to paragraph 7 which comprises determining whether the polynucleotide obtained from said bacterium hybridises to one or more of a plurality of polynucleotide probes corresponding to one or more of the said regions.

9. A method according to paragraph 8 wherein the plurality of polynucleotide probes are present as a microarray.

10. A method according to any one of paragraphs 1 to 5 wherein the nucleotide sequence analysis step comprises an amplification step using one or more primers, at least one of which hybridises specifically to a sequence which differs between types.

11. A method according to any one of paragraphs 1 to 6 wherein the nucleotide sequence analysis step comprises an amplification step using primer pairs, at least one of which hybridise specifically to a sequence which differs between types.

12. A method according to paragraph 10 or paragraph 11 wherein said primers are selected from the primers shown in Table 2.

13. A method of typing a group B streptococcal bacterium which method comprises determining the presence or absence in the genome of said bacterium of one or more surface protein genes selected from rib, alp2 or alp3 genes.

14. A method according to paragraph 13 wherein determining the presence or absence of said surface protein genes comprises determining whether a polynucleotide obtained from said bacterium selectively hybridises to a polynucleotide probe corresponding to a region of said surface protein genes.

15. A method according to any one of paragraph 13 wherein determining the presence or absence of said surface protein genes comprises an amplification step using one or more primers which amplify specifically a region of said surface protein genes.

16. A method according to paragraph 15 wherein said primers are selected from the primers shown in Table 6.

17. A method according to any one of paragraphs 1 to 12 which further comprises determining the presence or absence in the genome of said bacterium of one or more surface protein genes selected from rib, alp2 or alp3 genes.

18. A method of typing a group B streptococcal bacterium which method comprises determining the presence or absence in the genome of said bacterium of one or more mobile genetic elements selected from IS861, IS1548, IS1381, ISSa4 and GBSi1.

19. A method according to paragraph 18 wherein determining the presence or absence of said mobile genetic elements comprises determining whether a polynucleotide obtained from said bacterium selectively hybridises to a polynucleotide probe corresponding to a region of said mobile genetic elements.

20. A method according to any one of paragraph 18 wherein determining the presence or absence of said mobile genetic elements comprises an amplification step using one or more primers which amplify specifically a region of said mobile genetic-elements.

21. A method according to paragraph 20 wherein said primers are selected from the primers shown in Table 10.

22. A method according to any one of paragraphs 13 to 17 which further comprises determining the presence or absence in the genome of said bacterium of one or more mobile genetic elements selected from IS861, IS1548, IS1381, ISSa4 and GBSi1.

23. A polynucleotide consisting essentially of at least 10 contiguous nucleotides corresponding to a region within a cpsD-cpsE-cpsF-cpsG gene of a group B streptococcal bacterium, said polynucleotide comprising one or more nucleotides which differ between group B streptococcal serotypes.

24. A polynucleotide according to paragraph 23 wherein said nucleotides which differ between group B streptococcal serotypes correspond to one or more of positions 62, 78-86, 138, 139, 144, 198, 204, 211, 281, 240, 249, 300, 321, 419, 429, 437, 457, 466, 486, 602, 606, 627, 636, 645, 803, 971, 1026, 1044, 1173, 1194, 1251, 1278, 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

25. A polynucleotide consisting essentially of at least 10 contiguous nucleotides corresponding to a region within a sequence delineated by the 3′ 136 base pairs of cpsE and the 5′ 218 base pairs of cpsG of the cpsE-cpsF-cspG gene cluster of a group B streptococcal bacterium, said polynucleotide comprising one or more nucleotides which differ between group B streptococcal types.

26. A polynucleotide according to paragraph 25 wherein said nucleotides which differ between group B streptococcal types correspond to one or more of positions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

27. A polynucleotide consisting essentially of at least 10 contiguous nucleotides corresponding to a region within a cpsI/M gene of a group B streptococcal bacterium, said polynucleotide comprising one or more nucleotides which differ between streptococcal serotypes.

28. A polynucleotide according to paragraph 27 wherein the polynucleotide is selected from the nucleotide sequences shown in Table 2.

29. A polynucleotide consisting essentially of at least 10 contiguous nucleotides corresponding to a region within a rib, alp2 or alp3 gene of a group B streptococcal bacterium, said polynucleotide comprising one or more nucleotides which differ between group B streptococcal subtypes.

30. A polynucleotide according to paragraph 29 wherein the polynucleotide is selected from the nucleotide sequences shown in Table 6.

31. Use of a polynucleotide according to any one of paragraphs 23 to 30 in a method of serotyping and/or subtyping a group B streptococcal bacterium.

32. A composition comprising a plurality of polynucleotides according to any one of paragraphs 23 to 30.

33. Use of a composition according to paragraph 32 in a method of serotyping and/or subtyping a group B streptococcal bacterium.

34. A microarray comprising a plurality of polynucleotides according to any one of paragraphs 23 to 30.

35. Use of a microarray according to paragraph 34 in a method of serotyping and/or subtyping a group B streptococcal bacterium.

We claim:

1. A. method of typing a group B streptococcal bacterium, wherein the method comprises analysing the nucleotide sequence of one or more regions of a gene selected from the group consisting of cpsD, cpsE, cpsF. cpsG and cpsI/M genes of said bacterium, said region(s) comprising one or more nucleotides whose sequence varies between types of group B streptococcal bacteria.

2. The method according to claim 1, wherein the nucleotide sequence is analysed at one or more of positions 62, 78-86, 138, 139, 144, 198, 204, 211, 281, 240, 249, 300, 321, 419, 429, 437, 457, 466, 486, 602, 606; 627, 636, 645, 803, 971, 1026, 1044, 1173, 1194, 1251, 1278, 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 or 2196 as shown in FIG. 1.

3. The method according to claim 1, wherein at least one region is within a sequence delineated by the 3′ 136 bases of the cpsE gene and the 5′ 218 bases of cpsG of a cpsE-cpsF-cspG gene cluster of said streptococcal bacterium.

4. The method according to claim 3, wherein the nucleotide sequence is analysed at one ormore ofpositions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 or 2196 as shown in FIG. 1.

5. The method according to claim 1, wherein at least one region is within the cpsI/M genes of said bacterium.

6. The method according to claim 1, wherein analyzing the nucleotide sequence comprises sequencing said one or more regions.

7. The method according to claim 1, wherein analyzing the nucleotide sequence comprises determining whether a polynucleotide obtained from said bacterium selectively hybridises to a polynucleotide probe comprising one or more of the said regions.

8. The method according to claim 1, wherein analyzing the nucleotide sequence comprises determining whether the polynucleotide obtained from said bacterium selectively hybridises to one or more of a plurality of polynucleotide probes corresponding to one or more of the said regions.

9. The method according to claim 8, wherein the plurality of polynucleotide probes is present as a microarray.

10. The method according to claim 1, wherein analyzing the nucleotide sequence comprises an amplification step using one or more primers, at least one of which hybridises specifically to a sequence which differs between types of group B streptococcal bacteria.

11. The method according to claim 1, wherein analyzing the nucleotide sequence comprises an amplification step using primer pairs, at least one of which hybridises specifically to a sequence which differs between types of group B streptococcal bacteria.

12. The method according to claim 10, wherein said primers are selected from the primers shown in Table 2.

13. The method according to claim 11, wherein said primers are selected from the primers shown in Table 2.

14. A method of typing a group B streptococcal bacterium, wherein the method comprises determining the presence or absence, in the genome of said bacterium, of one or more surface protein genes selected from the group consisting of rib, alp2 and alp3 genes.

15. The method according to claim 14, wherein determining the presence or absence of said surface protein genes comprises determining whether a polynucleotide obtained from said bacterium selectively hybridises to a polynucleotide probe corresponding to a region of said surface protein genes.

16. The method according to claim 14, wherein determining the presence or absence of said surface protein genes comprises an amplification step using one or more primers which amplify a region of said surface protein genes.

17. The method according to claim 16 wherein said primers are selected from the primers shown in Table 6.

18. The method according to claim 1, which further comprises determining the presence or absence, in the genome of said bacterium, of one or more surface protein genes selected from the group consisting of rib, alp2 and alp3 genes.

19. A method of typing a group B streptococcal bacterium, wherein the method comprises determining the presence or absence, in the genome of said bacterium, of one or more mobile genetic elements selected from the group consisting of IS861, IS1548, IS1381, ISSa4 and GBSi1.

20. The method according to claim 19, wherein determining the presence or absence of said mobile genetic elements comprises determining whether a polynucleotide obtained from said bacterium selectively hybridises to a polynucleotide probe corresponding to a region of said mobile genetic elements.

21. The method according to claim 19, wherein determining the presence or absence of said mobile genetic elements comprises an amplification step using one or more primers which amplify a region of said mobile genetic elements.

22. The method according to claim 21, wherein said primers are selected from the primers shown in Table 10.

23. The method according to claim 14, which further comprises determining the presence or absence, in the genome of said bacterium, of one or more mobile genetic elements selected from the group consisting of IS861, IS1548, IS1381, ISSa4 and GBSi1.

24. A polynucleotide consisting essentially of at least 10 contiguous nucleotides of a region within a cpsD-cpsE-cpsF-cpsG gene cluster of a group B streptococcal bacterium, said polynucleotide comprising one or more nucleotides which differ(s) between group B streptococcal serotypes.

25. The polynucleotide according to claim 24, wherein said nucleotides which differ between group B streptococcal serotypes are at one or more of positions 62, 78-86, 138, 139, 144, 198, 204, 211, 281, 240, 249, 300, 321, 419, 429, 437, 457, 466, 486, 602, 606, 627, 636, 645, 803, 971, 1026, 1044, 1173, 1194, 1251, 1278, 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

26. A polynucleotide consisting essentially of at least 10 contiguous nucleotides of a region within a sequence delineated by the 3′ 136 base pairs of cpsE and the 5′ 218 base pairs of cpsG of a cpsE-cpsF-cspG gene cluster of a group B streptococcal bacterium, said polynucleotide comprising one or more nucleotides which differ(s) between group B streptococcal types.

27. The polynucleotide according to claim 26, wherein said nucleotides which differ between group B streptococcal types correspond to one or more of positions 1413, 1495, 1500, 1501, 1512, 1518, 1527, 1595, 1611, 1620, 1627, 1629, 1655, 1832, 1856, 1866, 1871, 1892, 1971, 2026, 2088, 2134, 2187 and 2196 as shown in FIG. 1.

28. A polynucleotide consisting essentially of at least 10 contiguous nucleotides corresponding to a region within a cpsI/M gene of a group B streptococcal bacterium, said polynucleotide comprising one or more nucleotides which differ(s) between streptococcal serotypes.

29. The polynucleotide according to claim 28, wherein the polynucleotide is selected from the nucleotide sequences shown in Table 2.

30. A polynucleotide consisting essentially of at least 10 contiguous nucleotides of a region within a rib, alp2 or alp3 gene of a group B streptococcal bacterium, said polynucleotide comprising one or more nucleotides which differ(s) between group B streptococcal subtypes.

31. The polynucleotide according to claim 30, wherein the polynucleotide is selected from the nucleotide sequences shown in Table 6.

32. A composition comprising a plurality of polynucleotides according to any one of claims 24 to 31.

33. A microarray comprising a plurality of polynucleotides according to any one of claims 24 to 31.
